(19) United States
(12) Patent Application Publication
(10) Pub. No.: US 2015/0299758 A1
(43) Pub. Date: Oct. 22, 2015

(54) MAGNETIC NANOPARTICLES

(71) Applicant: Kanagawa Academy of Science and Technology, Kawasaki-shi, Kanagawa (JP)

(72) Inventors: Kenji YASUDA, Tokyo (JP); Hyonchol KIM, Kanagawa (JP); Hideyuki TERAZONO, Kanagawa (JP); Akihiro HATTORI, Tokyo (JP)

(73) Assignee: Kanagawa Academy of Science and Technology, Kawasaki-shi, Kanagawa (JP)

(21) Appl. No.: 14/355,731

(22) PCT Filed: Nov. 8, 2012
(86) PCT No.: PCT/JP2012/078993

§ 371 Date: Oct. 4, 2014

(30) Foreign Application Priority Data
Nov. 8, 2011 (JP) 2011-244059

(51) Int. Cl. C12Q 1/24; B05D 1/30; H01F 1/01

(52) U.S. Cl. CPC C12Q 1/24; H01F 1/01; B05D 1/30

(57) ABSTRACT

The present invention provides a method for producing a microscopic object comprising locating a microscopic structure, formed of an organic substance, to be used as a mold on a substrate, depositing an intended element on a surface of the organic structure by a vacuum vapor deposition method or the like, and then decomposing and thus removing the organic structure as the mold by an ultraviolet-ozone process or the like to obtain the microscopic structure formed of only an intended element.

### TECHNICAL FIELD

The present invention relates to a hollow microscopic object formed of a metal, a semiconductor or the like, and a method for purifying a substance using the same.

### BACKGROUND ART

A microstructure such as a microparticle or the like is widely used as a ruler for a nanometer-sized structure, a material for a novel device, or in the field of biology, as a labeled substance for visualizing protein or DNA. Producing uniform-sized microparticles of various materials is indispensable to promote research and development in the above-described fields. The present inventors proposed a method for producing a microscopic object usable to label a biological molecule and a detection method using an electron microscope in Japanese Laid-Open Patent Publication No. Hei 11-001703 titled “Method for preparing ultramicroparticles” (Patent Document 1) and Japanese Laid-Open Patent Publication No. 2006-153826 titled “Biological sample labeled substance, biological substance labeling method, and method for inspecting biological substance” (Patent Document 2).

Japanese Laid-Open Patent Publication No. Hei 11-001703 discloses a method of forming microparticles by dispersing polystyrene spheres in the form of a layer on a flat substrate and vapor-depositing a metal or a semiconductor thereon. Japanese Laid-Open Patent Publication No. 2006-153826 discloses a method of immobilizing biological molecules of protein, DNA or the like to produced microparticles and a method of specifying the type of a surface-covered element by observation using a scanning electron microscope.

However, the microparticles produced by any of these methods are polystyrene or glass particles covered with an element. None of these publications describes a method for producing pure microparticles formed of only an intended element.

In the meantime, the great technological innovation which has enabled a single molecule to be observed has brought detailed information that was not available conventionally to various fields of physics, and has enabled many surprising phenomena to be discovered. A nanometer-sized light emitting element is usable as a fluorescent marker for tracing many physical and chemical elementary processes. For example, a nanometer-sized light emitting element attached to a large biological molecule allows a structural change or a molecular function thereof to be observed directly. Such a nanometer-sized light emitting element can also trace the movement of individual molecules and thus is usable to investigate a dynamic behavior of a biological cell. The extremely high potential of such observation of a single molecule is often limited by the following two factors. One factor is blinking. When blinking occurs during observation, the work of deriving useful information from the experimental results is made complicated. The other factor is that measurement is limited by photobleaching. Molecules in an excited state may occasionally cause a non-reversible chemical reaction by an extra energy thereof, resulting in not emitting fluorescence.

Since the development of a light emitting element that is not easily bleached, studies on the blinking phenomenon on a long-time scale have been performed. This nanometer-sized light emitting element, which is considered to perform measurement for an infinite time duration, is a semiconductor nanocrystal and is generally called a “colloidal quantum dot”. “Quantum dot” is one of the most brilliant results generated by nanotechnology, and electrical and optical properties thereof provided by the size thereof are highly attractive (A. P. Alivisatos, Science 271, 933 (1996) (Non-patent Document 1)).

Blinking of fluorescence from an isolated quantum dot was first observed in 1996 by cooperative research of Moungi Bawendi of MIT and Louis Brus of Bell Laboratories (in 1996) (M. Nirmal, B. O. Dabbousi, M. G. Bawendi, J. J. Macklin, J. K. Trautman, T. D. Harris, L. E. Brus, Nature 383, 802 (1996) (Non-patent Document 2)).

In fact, nothing was known at the time on what influences the light emission of dots. Therefore, blinking of quantum dots was a great surprise. In addition, the research group had confirmed that the time durations in which the quantum dots stay in an ON-state or an OFF-state (stay time durations) are not distributed exponentially. This result suggests that there is a complicated process behind this newly found blinking phenomenon. Later, a group including Masaru Kuno and David Nesbitt of JILA found that the probability density distribution of the ON-time and OFF-time conforms to the following power law (M. Kuno, D. P. Fromm, H. F. Hamann, A. Gallagher, D. J. Nesbitt, J. Chem. Phys. 115, 1028 (2001) (Non-patent Document 3)).

P(t)∝t^{-α}, 1<α<2 [Expression 1]

In the expression, t is the length of the ON-time or OFF-time, and the exponent α is between 1 and 2 (typically, 1.5).

This power law has been confirmed with almost all randomly located single quantum light emitting elements formed of semiconductor nanorods or nanowires, organic molecules, fluorescent proteins, composite polymers, and the like.

Now, as an example of colloidal quantum dot which has been recently used as a fluorescence source, a colloidal quantum dot such as CdSe will be described. A CdSe quantum dot having a size of about 2 to 6 nm can be chemically synthesized. Such quantum dots are each an isolated semiconductor nanocrystal. The size range of about 2 to 6 nm is the size range at which various property values change from those of a molecule to those of a bulk. Where the size of the nanocrystal is smaller, the area in which the charge carrier can move around is narrower. This is called the “quantum confinement effect”. As a result of this effect, a smaller quantum dot has discrete energy levels and a large bandgap. When being irradiated with light of energy larger than the bandgap, the quantum dot absorbs the light and forms an electron-hole pair called an “exciton”. This exciton emits light and is extinguished later. By changing the size or composition of the dot, the optical absorbance edge or the light emitting wavelength can be freely controlled in a visible light range. CdTe quantum dots (particle diameter: 2.5 nm to 5 nm), CdHgTe quantum dots, HgTe quantum dots and the like have been put into practice and confirmed to emit various types of fluorescence.

Since a colloidal quantum dot is extremely small, there are many dangling bonds, namely, unpaired electrons, which are present on a surface or in the vicinity of a defect of a covalent-bindable substance and are not involved in binding, are on a surface of the colloidal quantum dot. Excited electrons are captured by the dangling bonds, and therefore the performance of the quantum dot is deteriorated. Currently, it is being attempted to modify the surface of a quantum dot with an organic ligand, and two effects thereof have been confirmed. One is that the colloidal quantum dot is allowed to stably exist in the state of being dispersed in a solution, and the other is that the influence of the dangling bonds can be alleviated. According to another technique for occluding the dangling bonds on the surface, the surface is covered with an inorganic shell. For the shell, a semiconductor having a large bandgap is often used. Such a quantum dot is called a “core-shell quantum dot”.

Studies on what causes the behavior of blinking conforming to the power law has not progressed much. However, two new techniques regarding the blinking phenomenon have been recently developed. One technique was obtained while it was attempted to add a new function of the quantum dot for the purpose of using the quantum dot for a different application. When various molecular ligands were bound to the surface of a quantum dot, the optical properties of the dot were significantly changed (V. Fomenko, D. J. Nesbitt, Nano Lett. 8, 287 (2008) (Non-patent Document 4)). For example, a ligand acting as an electron donor dramatically decreases the length of the OFF-time and the frequency at which the OFF-time appears. However, this does not necessarily mean that the dynamics of blinking are changed at the same time. Although the frequency at which the OFF-time appears is decreased, the blinking still conforms to the power law. Therefore, this technique has brought a great advancement for a realistic application of decreasing the frequency of blinking, that is not very desirable.

According to the other technique, the CdSe quantum dot is used as a core and is covered with a thick (5 to 15 nm) shell of CdS crystal to suppress the blinking (B. Mahler, P. Spinicelli, S. Buil, X. Quelin, J.-P. Hermier, B. Dubertret, Nat. Mater. 7, 659 (2008) (Non-patent Document 5); Y. Chen, J. Vela, H. Htoon, J. L. Casson, D. J. Werder, D. A. Bussian, V. I. Klimov, J. A. Hollingsworth, J. Am. Chem. Soc. 130, 5026 (2008) (Non-patent Document 6)). From this discovery, it is understood that blinking that conforms to the power law is related to a defect around the quantum dot and is not caused by the crystal itself In fact, it is already well known that fluorescence emitted by a so-called self-organized quantum dot, the lattice constant of which is matched almost perfectly with that of a substrate formed spontaneously under a certain condition while a high quality crystal is epitaxially grown, does not blink.

It is suggested theoretically and from the experimental results that separating quantum dots by a certain distance or longer, not locating quantum dots three-dimensionally in a dispersed manner, covering each of the quantum dots so that the quantum dots are not ionized, for example, are effective to prevent the blinking of the quantum dots.

In the meantime, artificially constructing a nano-space by which quantum dots can be isolated from an external area is expected to be useful for various purposes including selective release of a caged compound by an optical stimulus, and reproduction of protein by incorporation of denatured protein into chaperon protein. Nonetheless, technology for constructing an isolated space effectively and in a reversible manner by an artificial nano-structure has not been developed.

In light of such a situation, the present inventors have already developed a microscopic object that has a hollow structure that is formed of only a layer structure containing a metal, a transition metal or a semiconductor having a desired thickness, and a method for producing the same (Japanese Laid-Open Patent Publication No. 2011-101941 (Patent Document 3)). This provides microscopic objects of a uniform particle diameter that are formed of only an intended element and include quantum dots having a particle diameter as uniform as that of the organic molds.

Recently, especially in cancer research, extraction of cancer cells in the blood is a target of attraction as a technique for early diagnosis. There are cases where the size of cancer cells circulating in the blood is larger than the size of common leukemia cells in the blood. Therefore, for actually recovering the cancer cells, the usefulness of a cell recovery method that pays attention to the size of the cells, and a cell recovery method that uses antibody bound to a surface antigen specific to the cancel cells, are being examined. According to a technique for purifying a specific cell, a membrane filter having microscopic pores of a certain size that is available from Millipore Corporation is used, and size fractionation is performed based on whether or not the cells are sufficiently small to pass the pores. This technique has problems that, for example, the pores are clogged, the cells are damaged by a pressure applied thereto, and cells larger than the pores can be captured but smaller cells cannot be recovered. According to another technique, microparticles having antibody attached to surfaces thereof are used, and target cells bound to the antibody are recovered by recovering the microparticles. Especially according to a magnetic bead method developed and put into practice by DYNAL, ferromagnetic microscopic particles of iron or the like that are pulverized to a size too small to construct a magnetic domain structure are mixed as superparamagnetic particles into several-micrometer polystyrene microparticles to produce magnetic beads. The magnetic beads generate a ferromagnetic property only when an external magnetic field is provided by a magnet or the like. When the external magnetic field is removed, the microscopic ferromagnetic particles, which are each too small to construct a magnetic domain structure and therefore cannot maintain the ferromagnetic property and behave as paramagnetic particles. As a result, the magnetic beads, which have been agglutinated, are dispersed. This technique, when combined with antibody, can recover the cells bound to the antibody, but cannot recover a target in accordance with the size of the cells or molecules.

### CITATION LIST

### Patent Literature

Patent Document 1: Japanese Laid-Open Patent Publication No. Hei 11-001703

Patent Document 2: Japanese Laid-Open Patent Publication No. 2006-153826

Patent Document 3: Japanese Laid-Open Patent Publication No. 2011-101941

### Non-Patent Literature

Non-patent Document 1: A. P. Alivisatos, Science 271, 933 (1996)

Non-patent Document 2: M. Nirmal, B. 0. Dabbousi, M. G. Bawendi, J. J. Macklin, J. K. Trautman, T. D. Harris, L. E. Brus, Nature 383, 802 (1996)

Non-patent Document 3: M. Kuno, D. P. Fromm, H. F. Hamann, A. Gallagher, D. J. Nesbitt, J. Chem. Phys. 115, 1028 (2011)

Non-patent Document 4: V. Fomenko, D. J. Nesbitt, Nano Lett. 8, 287 (2008)

Non-patent Document 5: B. Mahler, P. Spinicelli, S. Buil, X. Quelin, J.-P. Hermier, B. Dubertret, Nat. Mater. 7, 659 (2008)

Non-patent Document 6: Y. Chen, J. Vela, H. Htoon, J. L. Casson, D. J. Werder, D. A. Bussian, V. I. Klimov, J. A. Hollingsworth, J. Am. Chem. Soc. 130, 5026 (2008)

### SUMMARY OF INVENTION

### Technical Problem

Therefore, a method for recovering, selectively and/or effectively, a target microparticle of a specific size such as a cell or the like that replaces filter fractionation is desired.

### Solution to Problem

In light of the above situation, the present invention provides a microscopic object that has a hollow structure formed of only a layer structure containing a metal, a transition metal or a semiconductor of a desired thickness and also contains microparticles that are formed of a substance exhibiting a ferromagnetic property or a ferrimagnetic property at room temperature and have a size too small to maintain a magnetic domain structure; a microscopic object including a cell-bindable substance attached to an inner surface of an innermost layer of the hollow structure; a method for producing the same; and a cell recovery method using the same.

In one embodiment, the microparticles are located two-dimensionally in a dispersed manner at an interface between two specific substance layers among a plurality of substance layers located inside the microscopic object having the hollow structure. The microparticles contain a fluorescence-emitting substance.

In another embodiment of the present invention, one of the metal layers is formed of a substance exhibiting a ferromagnetic property or a ferrimagnetic property at room temperature and has a thickness too small to maintain a magnetic domain. In addition, a method for producing such a microscopic object; a microscopic object including a cell-bindable substance attached to an inner surface of an innermost layer of the hollow structure; and a method for purifying a microparticle such as a cell or the like using the microscopic object are provided.

Examples of a single-element substance exhibiting a ferromagnetic property at room temperature include iron, cobalt, nickel, gadolinium and the like. Examples of a single-element substance exhibiting a ferrimagnetic property at room temperature include magnetic oxides referred to as “ferrites” such as FeO.Fe₂O₃, MnO.Fe₂O₃, NiO.Fe₂O₃, CoO.Fe₂O₃ and the like; and insulating ferrimagnetic materials such as iron garnet (garnet) M₃.Fe₅O₁₂ (M represents an element of Fe, Y, Mn or the like), for example, yttrium iron garnet (YIG) Y₃.Fe₅O₁₂ and the like.

In still another embodiment of the present invention, a method for producing a microscopic object described below is provided. Among the plurality of metal layers, an inner layer is formed of a substance exhibiting a ferromagnetic property or a ferrimagnetic property at room temperature and has a thickness too small to maintain a magnetic domain structure. A layer immediately outer thereto is formed of a substance exhibiting an insulating property at room temperature (insulator). A layer immediately outer thereto is also formed of a substance exhibiting a ferromagnetic property or a ferrimagnetic property at room temperature and has a thickness too small to maintain a magnetic domain structure. Such a layer structure including the magnetic substances and the insulator is stacked repeatedly N times (at least twice). The microscopic object produced by this method has a magnetic moment which is N times as large as the magnetic moment of a microscopic object including a single magnetic layer. The magnetic moment is N times as large in accordance with the number of the layer structures stacked. In addition, a microscopic object including a cell-bindable substance attached to an inner surface of an innermost layer of the hollow structure; and a method for purifying a microparticle such as a cell or the like using the microscopic object are provided.

Herein, N represents a finite number of times of repetition, and is 2 or more.

Herein, the “insulator” refers to a substance commonly used in the field (substance having an electric conductivity σ of about 10⁻⁶ S/cm or less at room temperature), and is typically silicon oxide, silicon dioxide or the like.

In still another embodiment, the surface of the microscopic object is soluble by change in a property of an environmental liquid; a macromolecule bound to the inner surface of the microscopic object is extendable or foldable by change in a property of an environmental liquid, electric and/or magnetic field application, light irradiation or the like; and/or both of two ends of the macromolecule are bound to inner surfaces of two or more hollow microscopic objects, so that the two hollow microscopic objects can be joined together by change in a property of an environmental liquid.

More specifically, the present invention provides the following hollow microscopic object, a method for producing the same, and a use thereof.

(1) A hollow microscopic object comprising: a layer structure which forms an outer shell and comprises at least one thin film layer of a transition metal, a metal or a semiconductor; and an inner space and an opening defined by the layer structure, wherein:
the layer structure includes:
   at least two thin film layers; and
   microparticles embedded two-dimensionally in a dispersed manner at an interface between the at least two thin film layers; and
the microparticles are formed of a substance which exhibits a ferromagnetic property or a ferrimagnetic property at room temperature or a quantum dot, have a size too small to maintain a magnetic domain structure and are formed of a substance different from that of the thin film layers.

(2) A hollow microscopic object comprising: a layer structure which forms an outer shell and comprises at least one thin film layer of a transition metal, a metal or a semiconductor; and an inner space and an opening defined by the layer structure, wherein:
the layer structure includes at least two thin film layers; and
at least one of the layers is formed of a substance exhibiting a ferromagnetic property or a ferrimagnetic property at room temperature and has a thickness too small to maintain a magnetic domain structure.

(3) A hollow microscopic object comprising: a layer structure which forms an outer shell and comprises at least one thin film layer of a transition metal, a metal or a semiconductor; and an inner space and an opening defined by the layer structure, wherein:
the layer structure includes at least three thin film layers; and
at least two of the layers are formed of a substance exhibiting a ferromagnetic property or a ferrimagnetic property at room temperature and have a thickness too small to maintain a magnetic domain structure, and a layer that separates the at least two layers from each other is formed of an insulator and has a thickness that provides such a distance between the magnetic layers as not to generate a ferromagnetic property.

(4) The microscopic object according to (1), (2) or (3) above, wherein the transition metal has any of atomic numbers up to 79 except for 43, the metal has any of atomic numbers 13, 31, 32, 33, 49, 50, 51, 81, 82 and 83, and semiconductor has any of atomic numbers 14, 34 and 52, of the periodic table.

(5) The microscopic object according to any one of (1) through (4) above, wherein:
the substance which exhibits a ferromagnetic property at room temperature is selected from the group consisting of iron, cobalt, nickel and gadolinium; and
the substance which exhibits a ferrimagnetic property at room temperature is selected from the group consisting of FeO.Fe₂O₃, MnO.Fe₂O₃, NiO.Fe₂O₃, CoO.Fe₂O₃, iron garnet (garnet) M₃.Fe₅O₁₂ (M represents an element of Fe, Y, Mn or the like), and yttrium iron garnet (YIG) Y₃.Fe₅O₁₂.

(6) The microscopic object according to any one of (1) through (5) above, wherein:
(i) an outermost layer of the layer structure is formed of gold and has a thickness of 2 nm or more; or (ii) an innermost layer of the layer structure is formed of gold and has a thickness of 2 nm or more.

(7) The microscopic object according to (3) above, wherein:
(i) the layers separated from each other by the insulator which does not have a ferromagnetic property or a ferrimagnetic property at room temperature each have a thickness of 10 nm or more; or
(ii) the layers separated from each other by the insulator which does not have a ferromagnetic property or a ferrimagnetic property at room temperature are each formed of an insulating substance or a metal oxide.

(8) The microscopic object according to (2) or (3) above, wherein a layer of the microparticles has a thickness of 5 nm or less.

(9) The microscopic object according to (1) above, wherein the quantum dot is selected from the group consisting of Cds, CdSe, CdTe, CdHgTe and HgTe.

(10) The microscopic object according to (1), (2) or (3) above, wherein:
the layer structure includes at least two thin film layers;
an outermost thin film layer of the layer structure is formed of a substance soluble by a predetermined liquid; and
an inner thin film layer of the layer structure is formed of a substance insoluble by the liquid.

(11) The microscopic object according to (10) above, wherein the outermost thin film layer is formed of a metal, and the inner thin film layer is formed of a dielectric substance or a semiconductor.

(12) The microscopic object according to (1), (2) or (3) above, further comprising a macromolecule having one of two ends thereof immobilized to a surface of the layer structure that is exposed to the inner space.

(13) The microscopic object according to (12) above, wherein an innermost layer and an outermost layer of the layer structure are formed of different substances from each other, the substance of the innermost layer is suitable to allow for the one end of the macromolecule to be attached thereto, and the substance of the outermost does not easily allow the one end of the macromolecule to be attached thereto.

(14) The microscopic object according to (12) or (13) above, wherein the innermost layer is formed of any one of gold, silver, silicon and silicon oxide, and the outermost layer is formed of any one of iron, copper, germanium, aluminum, chromium, tin, titanium, manganese, nickel, cobalt and gadolinium.

(15) The microscopic object according to any one of (12) through (14) above, wherein the macromolecule has a structure thereof changeable by change in ionic strength and/or pH of a solution, electric field application, magnetic field application, or light irradiation.

(16) The microscopic object according to any one of (12) through (15) above, wherein the macromolecule is a DNA chain or cellulose polymer, or those formed of a DNA chain or a cellulose polymer that are coupled to each other by a molecule having a structure thereof changeable by electric field application, magnetic field application or light irradiation.

(17) The microscopic object according to any one of (12) through (15) above, wherein the macromolecule is, for example, a nucleic acid molecule or molecular chain, a nucleic acid derivative molecule or molecular chain, a molecular chain of protein such as antibody or the like, a macromolecular chain bindable to a cell surface or the like.

(18) The microscopic object according to any one of (12) through (15) above, wherein the other end of the macromolecule is immobilized to another microscopic object or a surface of another substrate.

(19) A method for producing a hollow microscopic object, comprising the step of depositing at least one thin film layer of a transition metal, a metal or a semiconductor on a mold, which is a microstructure formed of an organic substance, to form a layer structure, wherein:
(i) the method further comprises, after the step of depositing at least one thin film layer of a transition metal, a metal or a semiconductor, the step of dispersing microparticles on a surface of the deposited layer to bind the microparticles thereto, and the step of, subsequently, depositing again at least one layer of a transition metal, a metal or a semiconductor, thereby embedding the microparticles two-dimensionally in a dispersed manner at an interface sandwiched between the substance deposited to form the first layer and the substance deposited to form the second layer; and
the microparticles are formed of a substance which exhibits a ferromagnetic property or a ferrimagnetic property at room temperature or a quantum dot, have a size too small to maintain a magnetic domain structure and are formed of a substance different from that of the thin film layers; or
(ii) the layer structure includes at least two thin film layers; and at least one of the layers is formed of a substance exhibiting a ferromagnetic property or a ferrimagnetic property at room temperature and has a thickness too small to maintain a magnetic domain structure; or
(iii) the layer structure includes at least three thin film layers; and at least two of the layers are formed of a substance exhibiting a ferromagnetic property or a ferrimagnetic property at room temperature and have a thickness too small to maintain a magnetic domain structure, and a layer that separates the magnetic layers from each other is formed of an insulator.

(20) A method for producing a hollow microscopic object containing a transition metal, a metal or a semiconductor, the method comprising the steps of:
dripping an organic mold suspension, containing organic molds having a predetermined diameter, an appropriate amount of pure water, and a material for suppressing a static repulsive force between the organic molds, onto one surface of a substrate to distribute the organic molds on the substrate at a predetermined density;
washing and thus removing an excessive amount of the organic molds that is not adsorbed to the substrate;
drying the organic molds distributed on the substrate;
cutting the organic molds to adjust a gap between the organic molds arranged on the substrate to a predetermined distance;
depositing at least one thin film layer of a transition metal, a metal or a semiconductor on the organic molds distributed on the substrate; and
decomposing and thus removing the organic molds having the at least one thin film layer of a transition metal, a metal or a semiconductor deposited thereon to obtain each of the hollow microscopic objects which are left above the substrate;
wherein:
(i) the method further comprises, after the step of depositing at least one thin film layer of a transition metal, a metal or a semiconductor, the step of dispersing microparticles on a surface of the deposited layer to bind the microparticles thereto, and the step of, subsequently, depositing again at least one layer of a transition metal, a metal or a semiconductor, thereby embedding the microparticles two-dimensionally in a dispersed manner at an interface sandwiched between the substance deposited to form the first layer and the substance deposited to form the second layer;
the microparticles are formed of a substance which exhibits a ferromagnetic property or a ferrimagnetic property at room temperature, has a size too small to maintain a magnetic domain structure and is different from that of the thin film layers; or
(ii) the thin film layer includes at least two thin film layers; and at least one of the layers is formed of a substance exhibiting a ferromagnetic property or a ferrimagnetic property at room temperature and has a thickness too small to maintain a magnetic domain structure; or
(iii) the layer structure includes at least three thin film layers; and at least two of the layers are formed of a substance exhibiting a ferromagnetic property or a ferrimagnetic property at room temperature and have a thickness too small to maintain a magnetic domain structure, and a layer that separates the magnetic layers from each other is formed of an insulator.

(21) The method according to (20) above, wherein the organic molds are cut by any one of a plasma etching process, an ion milling process, a converged ion beam process, and a resist process.

(22) The method according to (20) above, wherein the step of depositing at least one thin film layer of a transition metal, a metal or a semiconductor on the organic molds distributed on the substrate is performed by any one of a resistive heating vacuum vapor deposition method , a sputtering method, and a chemical vapor deposition method.

(23) The method according to (20) above, wherein the step of decomposing and thus removing the organic molds having the at least one thin film layer of a transition metal, a metal or a semiconductor deposited thereon is performed by any one of an ultraviolet-ozone process, a plasma decomposition process, a photocatalyst decomposition process, and a heating and incineration process.

(24) The method according to (20) above, wherein the step of obtaining each of the hollow microscopic objects further includes:
dripping a microscopic amount of liquid onto each of the hollow microscopic objects; and
while allowing ultrasonic waves to act on the other surface of the substrate, placing a member having a flat bottom surface on the one surface to which the hollow microscopic objects are immobilized such that a slight load is applied to the bottom surface, and moving the member in an optional direction to delaminate the microscopic objects from the substrate.

(25) The method according to (24) above, wherein the liquid dripped onto the hollow microscopic objects is pure water, or a combination of pure water and protein such as bovine serum, antibody or bovine serum albumin (BSA); synthetic DNA; or a surfactant such as citrate, phosphate, sodium dodecyl sulfate (SDS) or tannic acid.

(26) The method according to any one of (19) through (25) above, wherein the substrate is a silicon substrate, a glass substrate, an aluminum substrate, or a plastic substrate.

(27) A method for recovering a biological substance by use of a hollow microscopic object having an inner surface modified by a substance specifically bindable to a specific target biological substance and having a superparamagnetic property, the method comprising the steps of:
mixing the hollow microscopic object and a solution containing the target biological substance; and
attracting the substance that modifies the inner surface of the hollow microscopic object and is bindable to the target biological substance and also attracting the target biological substance bound thereto, by use of an external magnetic field, and recovering the substance and the target biological substance by a magnetic power.

(28) The method according to (27) above, wherein the biological substance is cell.

(29) The method according to (27) or (28) above, further comprising the step of, after the step of recovering, degrading the substance bound to the target biological substance and recovering the target biological substance.

(30) The method according to (29) above, wherein DNA aptamer is used as the substance bindable to the target biological substance, and a DNA degrading enzyme is used in the step of degrading.

(31) The method according to (27) above, further comprising the step of, after the step of recovering, degrading the hollow microscopic object.

(32) The method according to any one of (27) through (31) above, wherein the hollow microscopic object is a hollow microscopic object according to any one of (12) through (18) above.

(33) The method according to any one of (27) through (32) above, wherein the size of the hollow microscopic object is adjusted to allow size fractionation to be performed on the target biological substance.

### Advantageous Effects of Invention

According to the present invention, at least one layer of a metal, a transition metal or a semiconductor is stacked with a controlled thickness to produce microscopic objects of a uniform particle diameter. In order to allow the microscopic objects to be formed of only the element(s) of the stacked layer(s), organic molds are decomposed and thus removed by use of an active oxygen generation device or the like. As a result, microscopic objects formed of only the stacked element(s) are obtained. The obtained microscopic objects are acted on by weak vibration of ultrasonic waves or the like and thus can be dispersed in any solvent.

The present invention also provides a microscopic object including layers of a metal, a transition metal or a semiconductor having a desired thickness and also including quantum dots located between the layers, and a method for producing the same. As a result, blinking of fluorescence can be alleviated.

According to the present invention, microstructures of a uniform particle diameter that are formed of an organic substance such as polystyrene or the like are used as molds. At least one layer of a metal, a transition metal or a semiconductor is stacked with a controlled thickness on the molds, then quantum dots are located on the element deposited to form the at least one layer, and then at least one layer of a metal, a transition metal or a semiconductor is stacked thereon. After this, the organic molds are decomposed and thus removed by use of an active oxygen generation device or the like. As a result, microscopic objects formed of only the stacked element(s) can be obtained.

According to the present invention, microscopic objects formed of only an intended element(s) with no impurities such as polystyrene, glass or the like can be obtained. In the case where the organic molds have a uniform particle diameter, the resultant microscopic objects formed of only the intended element(s) have a particle diameter as uniform as that of the organic molds. With a conventional self-polymerization method or fracturing method, it is difficult to uniformize the particle diameter of the microscopic objects to such a degree when a certain element is contained. In the case where the organic molds have various shapes or sizes, the resultant microscopic objects formed of only the intended element(s) have various shapes or sizes. In addition, the present invention allows microparticles to be located two-dimensionally in a dispersed manner between layers of different substances of the microscopic object (namely, the microparticles are expanded in a direction parallel to the inter-layer interface).

In a microscopic object formed of only the intended element(s) according to the present invention, the quantum dots can have a particle diameter as uniform as that of the organic molds.

By use of a hollow microscopic object according to the present invention, a biological substance, such as a cell or the like, bound to an inner surface of the hollow microscopic object can be recovered in an easy and simple manner by use of an external magnetic field. Especially a biological substance, such as a specific cell or the like, that is smaller than the hollow microscopic object, namely, that can be accommodated in a hollow part of the hollow microscopic object can be selectively recovered. Therefore, size fractionation of a target biological substance such as a cell or the like is made possible. In the case where the microscopic object has a multiple magnetic layer structure, the recovery rate of the target biological substance such as a cell or the like can be freely controlled by adjusting the number of the magnetic layers, so that the target biological substance can be recovered at higher rate and selectively.

### BRIEF DESCRIPTION OF DRAWINGS

FIG. 1 provides conceptual views of a hollow microscopic object produced according to the present invention.

FIG. 2 provides conceptual views showing a procedure of producing a microscopic object.

FIG. 3 is a conceptual view of organic molds arranged on a substrate.

FIG. 4 provides scanning electron micrographs of organic molds; FIG. 4(a) is such a micrograph before a plasma etching process is performed; and FIG. 4(b) is such a micrograph after the plasma etching process is performed.

FIG. 5 is a conceptual view of organic molds having elements deposited thereon by a vacuum vapor deposition method.

FIG. 6 provides graphs showing results of optical absorbance spectrum measurement performed on unreacted DNA remaining in a supernatant after a microscopic object is reacted with DNA of a concentration of 3 μM that has a thiol group at a 5′-terminus; FIG. 6(a) shows the results in the case where the microscopic object is obtained by vapor-depositing a first layer of gold, germanium, copper or nickel to a thickness of 10 nm on a surface of a polystyrene spherical microscopic object having a diameter of 100 nm but without vapor-depositing a second layer of gold on the first layer; and FIG. 6(b) shows the results in the case where the microscopic object is obtained by vapor-depositing the second layer of gold to a thickness of 2 nm on the first layer; the line labeled as “Before” in each figure represents the result of the optical absorbance spectrum measurement performed on DNA having a concentration corresponding to 3 μM before the reaction with the microscopic object is performed.

FIG. 7 provides a table showing the results of calculation of the immobilization density of DNA containing a thiol group, the DNA being immobilized on a surface of a microscopic object that is obtained by vapor-depositing a first layer of gold, germanium, copper or nickel to a thickness of 10 nm on a surface of a polystyrene spherical microscopic object having a diameter of 100 nm and vapor-depositing a second layer of gold thereon to a thickness of 0, 2, 5 or 10 nm.

FIG. 8(a) is a conceptual view showing a procedure of performing an ultraviolet-ozone process on organic molds having elements deposited thereon; and FIG. 8(b) is a conceptual view after the organic molds are removed by the ultraviolet-ozone process.

FIG. 9(a) is a secondary electron image of produced iron microscopic objects observed by use of a scanning electron microscope; and FIG. 9(b) is a reflected electron image thereof.

FIG. 10(a) is a conceptual view of a microscopic object including quantum dots therein; FIG. 10(a′) is a conceptual view of the microscopic object after a layer of element is removed; and FIGS. 10(b) and 10(b′) are conceptual views showing wavelength characteristics of fluorescence emitted by the microscopic objects in FIGS. 10(a) and 10(a′) respectively.

FIG. 11(a) is a conceptual view showing a process of immobilizing biological molecules inside hollow microscopic objects and capturing target biological molecules by electric induction; FIG. 11(a′) is a conceptual view showing the target biological molecules captured as a result of the electric induction; FIG. 11(b) is a conceptual view showing a process of extending chain-like biological molecules to be straight and capturing target biological molecules inside hollow microscopic objects; FIG. 11(b′) is a conceptual view showing the target biological molecules captured by use of the chain-like biological molecules; FIG. 11(c) is a conceptual view showing a process of capturing target biological molecules in a space formed by two hollow microscopic objects coupled to each other by a chain-like biological molecule; and FIG. 11(c′) is a conceptual view of the target biological molecules captured in the space formed by the two hollow microscopic objects.

FIG. 12 shows particle states of hollow microscopic objects including a ferromagnetic layer having such a thickness as not to maintain a magnetic domain structure, before, during and after application of an external magnetic field.

FIG. 13 provides conceptual views schematically showing exemplary cell recovery methods that use a magnetic hollow microscopic object having a superparamagnetic property at room temperature.

FIG. 14(a) is a conceptual view of a hollow microscopic object including three thin film layers in total, which are two ferromagnetic layers each having such a thickness as not to maintain a magnetic domain structure and an insulating layer that separates the two layers from each other; and FIG. 14(b) is a graph showing the relationship between the number of ferromagnetic layers that have such a thickness as not to maintain a magnetic domain structure and are included in a microscopic object, and the responsiveness of the microscopic object to the magnetic field applied thereto.

### DESCRIPTION OF EMBODIMENTS

### 1. Hollow Microscopic Object According to the Present Invention

In one embodiment, the present invention provides a hollow microscopic object which has a layer structure including at least one thin film layer of a transition metal, a metal or a semiconductor and has an inner space and an opening defined by the layer structure.

The hollow microscopic object according to the present invention has a cap-like structure having an inner space and an opening defined by the layer structure. The cap part has a shape which may be changed in accordance with the shape of a mold used in a production process of the microscopic object, and may be hemispherical, cylindrical, conical, prism-shaped or the like with no specific limitation.

The hollow microscopic object according to the present invention has a size (or particle diameter) which may also be changed in accordance with the size of the mold used in the production process thereof and is selectable in accordance with a use thereof. The size of the hollow microscopic object according to the present invention is in the range of about 0.1 nm to about 1 mm, preferably in the range of about 1 nm to about 500 μm, more preferably in the range of about 5 nm to 100 μm, and most preferably in the range of about 5 nm to 1 μm.

In this specification, the single term “metal” refers to a metal of main group elements. The “main group elements” are elements in groups 1, 2, and 12 through 18 of the periodic table, and include all the non-metals and some of the metals. Preferable “metals” usable in the present invention are, for example, Al, Ga, Ge, As, In, Sn, Sb, Tl, Pb, Bi and the like.

In this specification, the term “transition metal” refers to a transition element, namely, an element of any of groups 3 through 11 in the periodic table. Preferable “transition metals” usable in the present invention are those having atomic numbers 21 through 79 except for 43.

In this specification, the term “semiconductor” is used in the meaning commonly used in the field (“substance having an electric conductivity σ of about 10³ to 10⁻¹⁰ S/cm, which is between the electric conductivity of a metal and the electric conductivity of an insulator, at room temperature”) (Iwanami Rikagaku Jiten, 5th ed., 1998, Iwanami Shoten Publishers). Preferable “semiconductors” usable in the present invention are, for example, Si, Se, Te and the like.

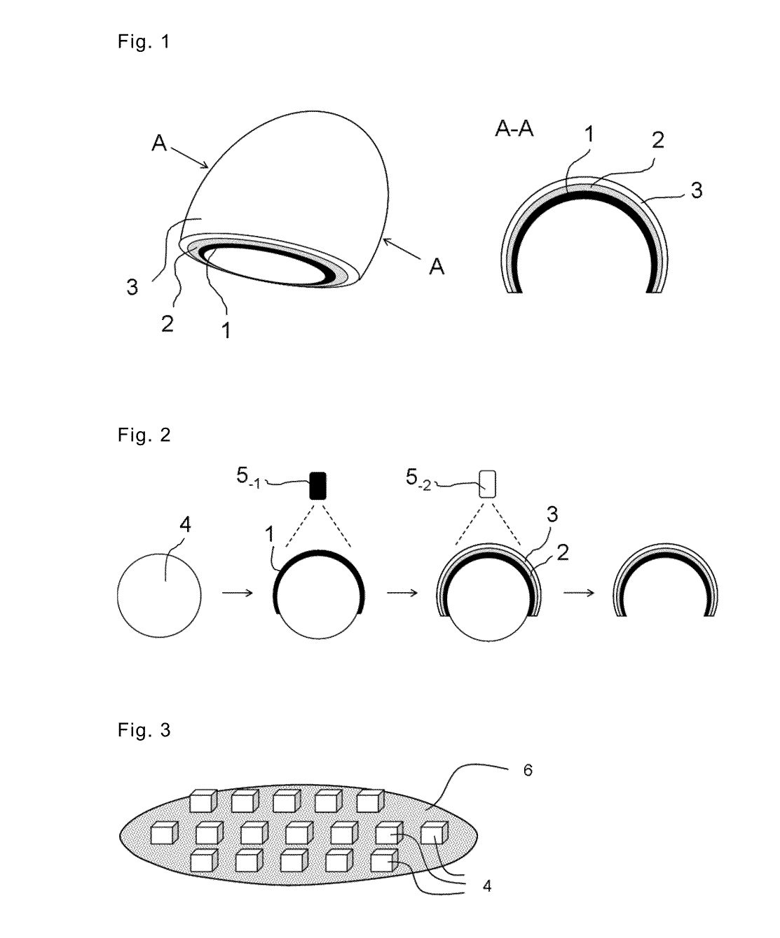

FIG. 1 shows an example of hollow microscopic object according to the present invention. In this example, a hemispherical hollow microscopic object including layers of three elements 1, 2 and 3. There is no limitation on the number of the layers or the shape of the microscopic object.

As shown in FIG. 1, an outer shell of the hollow microscopic object according to the present invention has a layer structure including a stack of thin film layers of the elements 1, 2 and 3. Each of the layers has a thickness that is typically in the range of about 1 nm to about 1 μm, more preferably in the range of about 1 nm to about 500 nm, and most preferably in the range of about 1 nm to about 100 nm. The thickness is not limited to such a range, and may be optionally set to a value suitable for a purpose in the range of about 0.1 nm as the lower limit to about 10 μm as the upper limit.

FIG. 2 schematically shows a method for producing a hollow microscopic object. An organic structure 4 having a size of, for example, 5 nm to 100 μm is used as a mold for the microscopic object. The elements 1, 2 and 3 emitted from vapor deposition sources 5₋₁ and 5₋₂ are sequentially deposited each to a thickness in the range of 1 nm to 500 nm on the organic structure 4 by a vacuum vapor deposition method, a sputtering method or the like. Then, the organic mold 4 is decomposed and thus removed by, for example, an ultraviolet-ozone process, a plasma decomposition process, a photocatalyst decomposition process, a heating and incineration process or the like. In this manner, the hollow microscopic object is obtained. A more specific procedure for producing the hollow microscopic object will be described later in this specification.

A hollow microscopic object provided in one embodiment according to the present invention includes a layer that is formed of an element or a compound having a ferromagnetic property or a ferrimagnetic property at room temperature and has a thickness too small to maintain a magnetic domain structure, and thus has a superparamagnetic property. Usable as such an element or compound are, for example, ferromagnetic single-substance elements exhibiting a ferromagnetic property at room temperature such as iron, cobalt, nickel, gadolinium and the like; magnetic oxides referred to as “ferrites” such as FeO.Fe₂O₃, MnO.Fe₂O₃, NiO.Fe₂O₃, CoO.Fe₂O₃ and the like; and ferrimagnetic materials including insulating ferrimagnetic materials such as iron garnet (garnet) M₃.Fe₅O₁₂ (M represents an element of Fe, Y, Mn or the like), yttrium iron garnet (YIG) Y₃.Fe₅O₁₂ and the like. The thickness too small to maintain a magnetic domain structure, which depends on the type of the substance, is generally 10 nm or less, 9 nm or less, 8 nm or less, 7 nm or less, 6 nm or less, preferably about 5 nm or less, 4 nm or less, 3 nm or less, 2 nm or less, or 1 nm or less. With such a thickness, a superparamagnetic property which cannot maintain a magnetic domain structure is realized.

A hollow microscopic object provided in another embodiment according to the present invention includes at least two layers that are formed of an element or a compound having a ferromagnetic property or a ferrimagnetic property at room temperature and have a thickness too small to maintain a magnetic domain structure, and also includes an insulating layer held between, to separate, the two layers, and thus has a superparamagnetic property. Elements or compounds usable to form the insulating layer are, for example, oxides such as SiO, SiO₂, manganese oxide, nickel oxide, alumina and the like; polymers such as Teflon (registered trademark) and the like; organic polymers such as plastics and the like; and insulators. The thickness of the insulating layer that separates the magnetic layers from each other, which depends on the type of the substance, is generally about 5 nm or more, and preferably about 10 nm or more. With such a thickness of the insulating layer, the multiple layer structure in which the magnetic layers are separated from each other by the insulating layer can have a superparamagnetic property.

A hollow microscopic object provided in still another embodiment according to the present invention has a layer structure including at least two thin film layers and includes microparticles embedded two-dimensionally in a dispersed manner at an interface between the at least two thin film layers. Such microparticles are formed of a substance different from the substance used to form the thin film layers.

In one embodiment, the particles are formed of a fluorescence-emitting substance. Such microparticles are, for example, microparticles (quantum dots) of a diameter of several nanometers that are formed of an element such as, for example, cadmium-selenium (CdSe) or the like. Examples of other substances usable for the quantum dots include CdS, CdTe, CdHgTe, HgTe and the like. In this embodiment, a certain ambient environment of the quantum dots can be maintained because the quantum dots are embedded in a dispersed manner at the interface between the layers. Therefore, the quantum dots can have a stable fluorescence characteristic. In this manner, the problem of the ionization of quantum dots can be solved.

In another embodiment, the microparticles may be formed of any of ferromagnetic single-substance elements exhibiting a ferromagnetic property at room temperature such as iron, cobalt, nickel, gadolinium and the like; magnetic oxides referred to as “ferrites” such as FeO.Fe₂O₃, MnO.Fe₂O₃, NiO.Fe₂O₃, CoO.Fe₂O₃ and the like; and ferrimagnetic materials including insulating ferrimagnetic materials such as iron garnet (garnet) M₃.Fe₅O₁₂ (M represents an element of Fe, Y, Mn or the like), yttrium iron garnet (YIG) Y₃.Fe₅O₁₂ and the like. When being used for the microparticles, such a substance may be put into a microscopic size too small to maintain a magnetic domain structure. The particle size is about 10 nm or less, 9 nm or less, 8 nm or less, 7 nm or less, 6 nm or less, preferably about 5 nm or less, 4 nm or less, 3 nm or less, 2 nm or less, or 1 nm or less. With such a particle size, a superparamagnetic property which cannot maintain a magnetic domain structure is realized.

In a preferable embodiment, the microparticles may be formed of a fluorescence-emitting substance described above and a substance exhibiting a ferromagnetic property or a ferrimagnetic property at room temperature described above. These substances may be used at the same time or in the form of a mixture.

A hollow microscopic object provided in still another embodiment according to the present invention further includes a macromolecule having one end thereof immobilized to a surface of the layer structure that faces the inner space. Examples of the “macromolecule” include biological molecules, for example, nucleic acid molecules (e.g., DNA, RNA), proteins, macromolecular polymers (e.g., cellulose polymer, polyethylene glycol) and the like. For a specific use in this embodiment, a macromolecule having a structure that is changeable in accordance with, for example, ionic strength or pH of a solution is preferable.

In the hollow microscopic object in this embodiment, an outermost layer of the shell contains a substance which does not easily allow one end of a macromolecule to be bound thereto such as, for example, iron, copper ,germanium, aluminum, chromium, tin, titanium, manganese, nickel or the like. An innermost layer of the shell contains a substance which easily allows one end of a macromolecule to be bound thereto such as, for example, gold, silver, silicon, silicon oxide or the like. Which substance is to be used may be easily determined by a person of ordinary skill in the art in accordance with the use of the hollow microscopic object.

A hollow microscopic object provided in still another embodiment according to the present invention has a layer structure which forms an outer shell and includes at least two thin film layers. An outermost thin film layer of the outer shell is soluble by a predetermined liquid, and an inner thin film layer thereof is insoluble by the liquid.

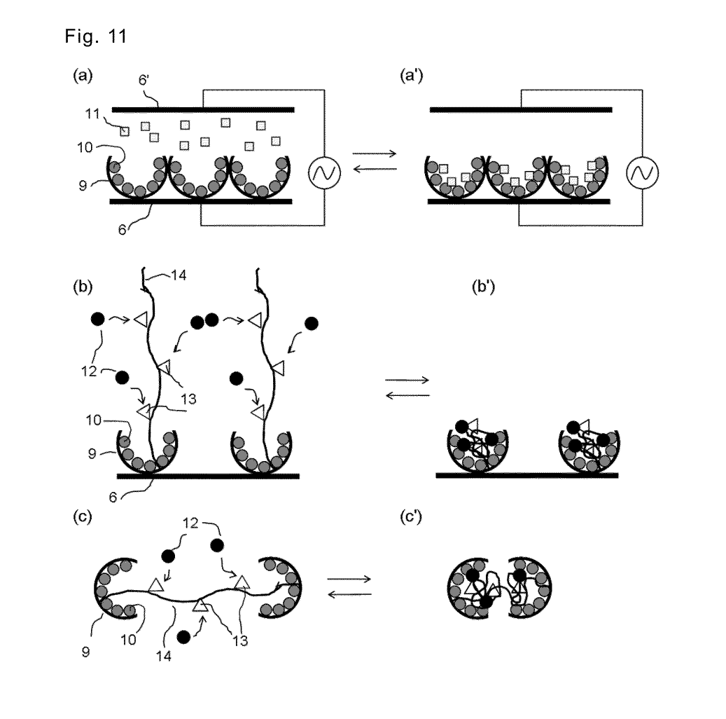

In this embodiment, for example, the outermost layer and an layer immediately inner thereto (referred to as “layer A”) are formed of a substance 2 that is soluble by a predetermined solution (e.g., aluminum, copper), and a layer immediately inner to the layer A (referred to as “layer B”) and a layer immediately inner to the layer B (referred to as “layer C”; may be the innermost layer) are formed of a substance 1 that is insoluble by the solution and is easily chemically modified (e.g., gold). Between the outermost layer and the layer A, quantum dots 8 of a nanometer size are embedded two-dimensionally in a dispersed manner. Between the layer B and the layer C, quantum dots 7 having optical characteristics different from those of the quantum dots 8 are embedded two-dimensionally in a dispersed manner. With such a form, a fluorescence resonance phenomenon occurs between the quantum dots 8 and 7, and fluorescence having a discriminative wavelength is stably emitted (see FIGS. 10(a) and (b)). In this manner, the problems of the blinking and the ionization of the quantum dots can be solved. The distance between the quantum dots 7 and 8 is appropriately adjustable such that the fluorescence resonance phenomenon occurs, by adjusting the thicknesses of the layer A and the layer B.

The hollow microscopic object in this embodiment according to the present invention may be exposed to a solution which can dissolve the substance 2 (the solution is, for example, hydrochloric acid), so that the outermost layer and the layer A immediately inner thereto are eluted and the quantum dots 8 are removed together with these layers. As a result, the fluorescence emitted from the microscopic object in this embodiment according to the present invention has a wavelength of the fluorescence which is emitted by the quantum dots 7. In this manner, a sensor which emits light of different wavelengths in accordance with solvent conditions can be provided (see FIGS. 10(a′) and (b′)).

The outermost thin film layer in this embodiment is preferably formed of a substance which can be eluted under a certain solvent condition, for example, a metal such as magnesium, aluminum or the like; a transition metal such as zinc, copper or the like; or a semiconductor such as germanium or the like; with no specific limitation. The innermost thin film layer is preferably formed of a substance which is easily chemically modified, for example, a transition metal such as gold, silver or the like; a dielectric material; or a semiconductor oxide such as silicon monoxide, silicon dioxide or the like; with no specific limitation. The “predetermined liquid” mentioned above varies in accordance with the substance used for the outermost layer. In the case where, for example, the outermost layer is formed of aluminum, the “predetermined liquid” may be hydrochloric acid or sodium hydroxide. In the case where the outermost layer is formed of copper, the “predetermined liquid” may be nitric acid. In the case where the outermost layer is formed of zinc, the “predetermined liquid” may be hydrochloric acid, sulfuric acid or the like.

### 2. Method for Producing a Hollow Microscopic Object According to the Present Invention

A method for producing a hollow microscopic object in one embodiment according to the present invention includes the step of depositing at least one thin film layer of a transition metal, a metal or a semiconductor on a mold, which is a microstructure formed of an organic substance.

More specifically, a method in an embodiment according to the present invention for producing a hollow microscopic object containing a transition metal, a metal or a semiconductor includes the steps of:
dripping an organic mold suspension, containing organic molds having a predetermined diameter, an appropriate amount of pure water, and a material for suppressing a static repulsive force between the organic molds, onto one surface of a substrate and distributing the organic molds on the substrate at a predetermined density;
washing and thus removing an excessive amount of the organic molds that is not adsorbed to the substrate;
drying the organic molds distributed on the substrate;
cutting the organic molds to adjust a gap between the organic molds arranged on the substrate to a predetermined distance;
depositing at least one thin film layer of a transition metal, a metal or a semiconductor on the organic molds distributed on the substrate; and
decomposing and thus removing the organic molds having the at least one thin film layer of a transition metal, a metal or a semiconductor deposited thereon to obtain each of the hollow microscopic objects which are left above the substrate.

FIG. 3 is a schematic view showing an example in which organic structures, to be used as molds for producing the hollow microscopic objects according to the present invention, are arranged on a substrate. In this example, prism-shaped organic molds 4 are arranged on a flat substrate 6. The substrate is generally formed of silicon, glass, aluminum, plastics or the like, but may be formed of any material as long as the substrate is flat.

The organic molds 4 have a size which can be changed in accordance with the use. The size may be in the range of about 0.1 nm to about 1 mm, preferably in the range of about 1 nm to about 500 μm, more preferably in the range of about 5 nm to about 100 μm, and most preferably in the range of about 5 nm to about 1 μm. Typically, the size is about 5 nm to about 100 μm. The organic molds 4 may be spherical, cylindrical, conical, cubic or the like. The organic molds 4 may be formed of a material optionally selected from polystyrene, polypropylene, polyethylene, poly(methyl syloxane) (PDMS), poly(methyl methacrylate) resin (PMMA) and the like in accordance with a desired final shape of the microscopic object to be produced. In the case where, for example, the microscopic object is to be spherical, polystyrene is suitable. In the case where the microscopic object is to be hemispherical, cylindrical, conical or cubic, PDMS, PMMA or the like is suitable.

The organic molds 4 may be arranged on the substrate 6 by a method of cutting the organic thin film applied to the substrate, a method which uses a self-organized layer formation method, a method of controlling the dispersion force of the organic molds in the solvent or the like. For example, according to the method of controlling the dispersion force of the organic molds in the solvent, an appropriate amount of salt is added to a solvent having the organic molds dispersed therein to suppress the static repulsive force between the organic molds, and the solvent in this state is applied to the substrate. In this manner, the organic molds can be arranged on the substrate at a high density. The amount of the salt to be added varies in accordance with the size or the material of the organic molds. In the case where, for example, polystyrene spheres having a diameter of 100 nm are used as the molds, the organic mold solution and a 500 mM solution of salt may be mixed at a ratio of 1:2.

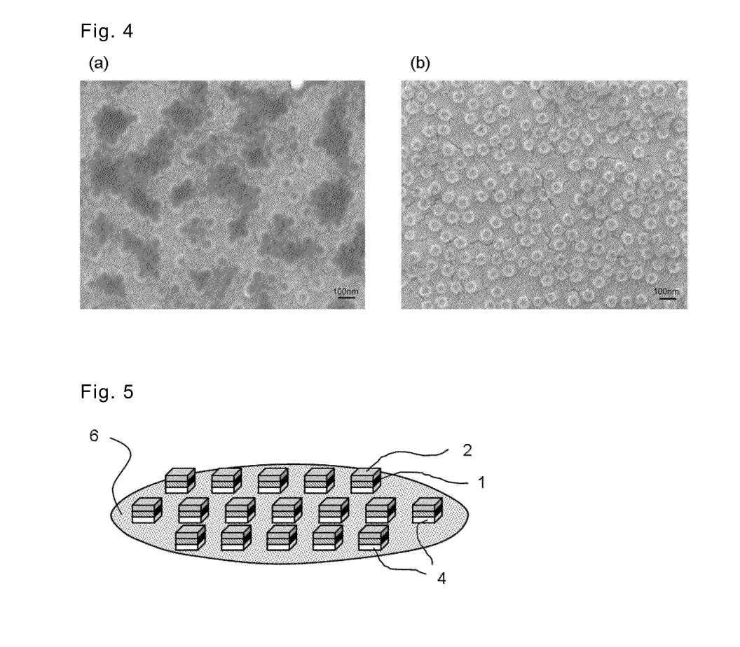

The size of, and the distance between, the organic molds 4 arranged on the substrate 6 can be optionally controlled by physical cutting such as a plasma etching process, an ion milling process, a converged ion beam process, a resist process or the like; or chemical cutting by use of an acidic solvent, an alkaline solvent or an organic solvent. As shown in, for example, FIG. 4, polystyrene spheres having a diameter of 100 nm that are arranged on the substrate so as to contact each other (see FIG. 4(a)) may be subjected to a plasma etching process for 15 seconds, so that the polystyrene spheres have a diameter of about 80 nm and are arranged at an interval of about 30 nm (see FIG. 4(b)).

After the organic molds 4 having an appropriate size are arranged on the substrate 6 at an appropriate interval, a desired element for the microscopic object to be produced is deposited on the molds. As the method for the deposition, an appropriate method may be selected from a (resistive heating) vacuum vapor deposition method, a sputtering method, a chemical vapor deposition method and the like. Hereinafter, a method for producing a microscopic object by a vacuum vapor deposition method will be described, for example.

Examples of the element usable as a vapor deposition source (element usable to form the microscopic object) are the following in the periodic table:
(1) transition metals having atomic numbers up to 79 except for 43;
(2) metals having atomic numbers 13, 31, 32, 33, 49, 50, 51, 81, 82 and 83; and
(3) semiconductors having atomic numbers 14, 34 and 52.

The substrate 6 and the organic molds 4 arranged thereon are put into a vacuum vapor deposition device, and at least one element layer is deposited. The deposition may be performed as follows, for example.

First, the substrate 6 and the organic molds 4 arranged thereon in advance are set in a chamber of the resistive heating vapor deposition device such that the organic molds 4 are directed toward a vapor deposition source in the device. The degree of vacuum in the camber is, for example, 5×10⁻⁴ pascal, and the temperature in the chamber is room temperature. The chamber accommodates a shutter provided between the organic molds 4 and the vapor deposition source. The vapor deposition source includes a vapor deposition source vessel and a heating resistor. The shutter is movable forward and backward or leftward and rightward. In the state where the shutter covers the entire surface of the substrate 6, vapor deposition to the organic molds is inhibited. In the state where the shutter is moved to expose the substrate 6 to the vapor deposition source, vapor deposition to the organic molds 4 is performed. The vapor deposition vessel accommodates a metal, a transition metal or a semiconductor to be vapor-deposited to surfaces of the organic molds. The heating resistor is used to heat and evaporate the element in the vapor deposition vessel.

FIG. 5 is a schematic view of two layers of the elements 1 and 2 deposited on the organic molds 4. In the case where at least two element layers are to be vapor-deposited, the elements may be deposited from the element that is to form the innermost surface. In the example of FIG. 5, the element 1 is vapor-deposited and then the element 2 is vapor-deposited. The post-vapor deposition thickness of each of the elements may be appropriately selected from the range of, for example, 1 nm to 500 nm.

Referring to FIG. 5, the layer corresponding to the element 2, namely, the outermost layer, among the layers to be deposited on the surface of the microscopic objects, may be formed of a material for which molecular modification by gold, silver or the like is easily performed, so that the organic molecules can be immobilized to the surface of the microscopic objects. For example, it is well known that a thiol group is strongly bound to a surface of gold. Utilizing this binding reaction, various types of organic molecules such as DNA and RNA having a thiol group at a terminus, protein such as, for example, antibody containing a thiol group, single molecule film-forming reagents containing a thiol group and the like can be immobilized. In order to allow the outermost element layer to be a stable immobilization layer, the thickness thereof is preferably at least about 1 nm, more preferably at least about 1.5 nm, and most preferably at least about 2 nm.

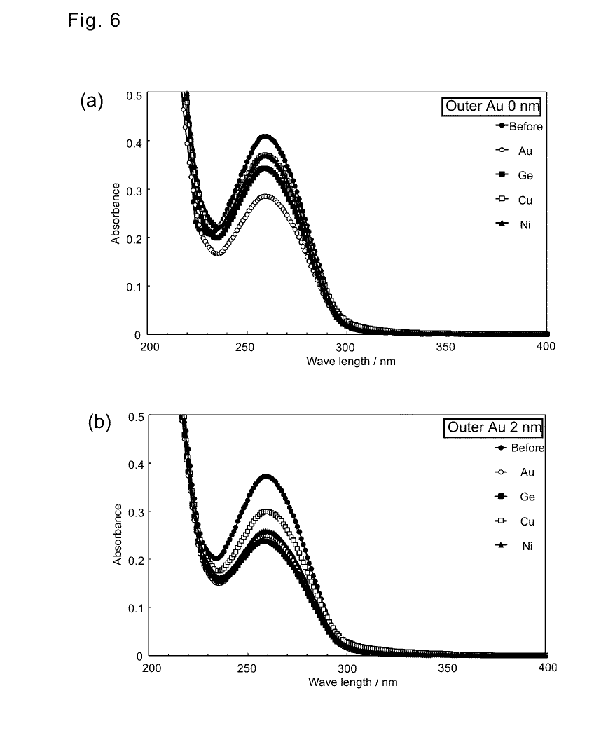

FIG. 6 will be described. On surfaces of polystyrene spherical microscopic objects having a diameter of 100 nm, first layers respectively of gold, germanium, copper and nickel were vapor-deposited to a thickness of 10 nm. In one test, a second layer of gold was not vapor-deposited on each first layer; and in the other test, the second layer of gold was vapor-deposited on each first layer. The resultant microscopic objects were each reacted with DNA of a concentration of 3 μM that has a thiol group at a 5′-terminus. Then, optical absorbance spectrum measurement was performed on the unreacted DNA remaining in the supernatant. FIG. 6(a) shows the results of the optical absorbance spectrum measurement in the case where the second layer of gold was not vapor-deposited, and FIG. 6(b) shows the results of the optical absorbance spectrum measurement in the case where the second layer formed of gold was vapor-deposited. The horizontal axis represents the wavelength of the light, and the vertical axis represents the optical absorbance. The figures also show the results of the optical absorbance spectrum measurement performed on the DNA having a concentration corresponding to 3 μM before the reaction with the microscopic object was performed. The DNA has an absorbance peak at 260 nm. Therefore, the number of DNA molecules immobilized to the surfaces of the microscopic objects can be quantified by calculating the difference in the optical absorbance at 260 nm before and after the reaction of immobilizing the DNA to the microscopic objects. In the graph of the case where gold is not present as the outer layer (FIG. 6(a)), in the case where the first layer is formed of germanium, copper or nickel, no remarkable change is seen in the optical absorbance at 260 nm before and after the reaction because the thiol group is not strongly bound to the surface of each element. By contrast, in the graph of the case where the second layer of gold is formed to a thickness of 2 nm as the outer layer (FIG. 6(b)), even in the case where the first layer is formed of germanium, copper or nickel, a change as remarkable as in the case where the first layer is formed of gold is seen in the optical absorbance at 260 nm before and after the reaction. Namely, it has been confirmed that DNA containing a thiol group can be immobilized at a high density by forming a gold layer having a thickness of 2 nm on the surfaces of the microscopic objects.

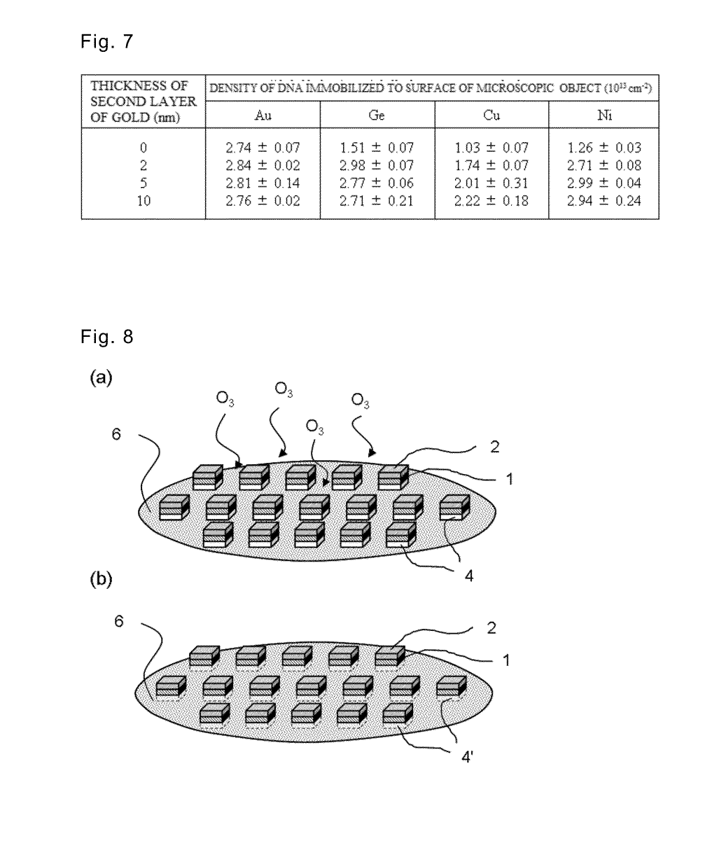

FIG. 7 will be described. On surfaces of polystyrene spherical microscopic objects having a diameter of 100 nm, first layers respectively of gold, germanium, copper and nickel were vapor-deposited to a thickness of 10 nm, and then second layers of gold were vapor-deposited on the first layers to thicknesses of 0, 2, 5 and 10 nm. The immobilization density of the thiol group-containing DNA immobilized to the surface of each microscopic object was calculated by differential measurement of the above-described optical absorbance spectrum. FIG. 7 provides a table showing the results of the calculation. The values obtained in the case where the first layer is formed of gold are used as controls. When the thickness of the second layer of gold is 2 nm, the immobilization density of the DNA obtained in the case where the first layer is formed of germanium or nickel is equivalent to the immobilization density of the DNA in the case where the first layer is formed of gold. Even when the thickness increases to 5 nm or to 10 nm, the immobilization degree does not change almost at all. Based on this, it has been confirmed that the second layer of gold acting as the outermost layer is sufficiently usable as a molecule immobilization layer when having a thickness of 2 nm or more and that even when the second layer is thicker, the performance is not changed.

In order to obtain intended microscopic objects, the organic molds may be decomposed and thus removed by either one of an ultraviolet-ozone process, a plasma decomposition process, a photocatalyst decomposition process, and a heating and incineration process. The method is not limited to one of these. In this example, as shown in FIG. 8, an ultraviolet-ozone process is used. The organic molds 4 having the elements 1 and 2 deposited thereon and the substrate 6 are put into a chamber of an ultraviolet-ozone decomposition device. Oxygen is introduced into the chamber and the inside of the chamber is irradiated with ultraviolet rays to generate ozone. As a result, the organic substances including the organic molds 4 are decomposed and thus removed. The time duration in which this process is performed is optionally adjustable by a person of ordinary skill in the art in accordance with the size of the organic molds or the like. In the case where the organic molds 4 are polystyrene spheres having a diameter of 100 nm, about 60 minutes is sufficient.

As a result of the decomposition and removal of the organic molds 4, hollow microscopic objects formed of only the vapor-deposited elements are obtained. In the example of FIG. 8, microscopic objects each having a two-layer structure of the elements 1 and 2 are obtained after the decomposition process. The produced microscopic objects may be delaminated from the substrate 6 by an ultrasonic process or the like and suspended in an appropriate solvent. The microscopic objects can be suspended in pure water by, for example, dripping pure water onto the substrate 6 and allowing ultrasonic waves to act on a surface of the substrate 6 opposite to the surface to which the microscopic objects are attached.

A method for producing a hollow microscopic object in a preferable embodiment according to the present invention may include, in addition to the above-described steps, the steps of:
dripping a microscopic amount of liquid onto the hollow microscopic objects; and
while allowing ultrasonic waves to act on the other surface of the substrate, placing a member having a flat bottom surface on the one surface to which the hollow microscopic objects are immobilized such that a slight load is applied to the bottom surface, and moving the member in an optional direction to delaminate the microscopic objects.

The liquid to be dripped onto the other surface of the substrate is not limited to pure water, and may be, for example, a combination of pure water and protein such as bovine serum, antibody, bovine serum albumin (BSA) or the like; synthetic DNA; or a surfactant such as citrate, phosphate, sodium dodecyl sulfate (SDS), tannic acid or the like. Alternatively, the liquid to be dripped onto the other surface of the substrate may be, for example, an acid such as sulfuric acid, hydrochloric acid, nitric acid or the like; an alkali such as ammonia, potassium hydroxide or the like; or an organic solvent such as ethanol, dimethylsulfoxide (DMSO) or the like.

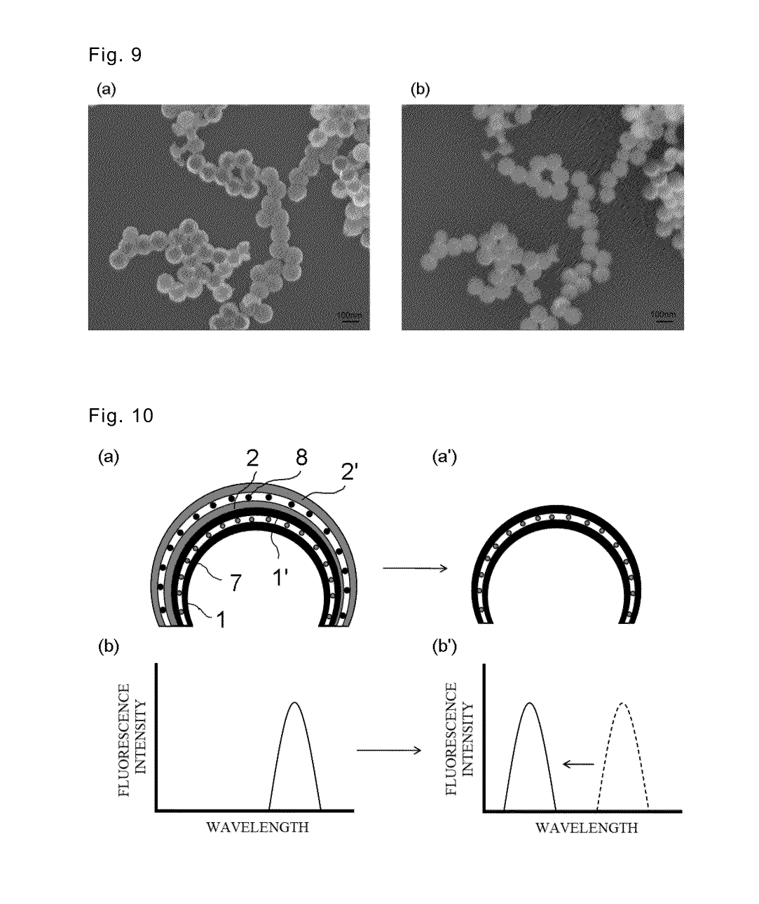

By the above-described procedure, hollow microscopic objects formed of only the vapor-deposited elements are obtained. FIG. 9 show an example of hollow microscopic objects thus obtained. FIG. 9 provides an example of microscopic objects observed by a scanning electron microscope. The microscopic objects shown in FIG. 9 are obtained by depositing iron to a thickness of 10 nm on polystyrene spherical molds having a diameter of 100 nm, decomposing and thus removing the organic molds by an ultraviolet-ozone process, suspending the resultant microscopic objects in pure water, and then arranging the microscopic objects again on another substrate. A detailed structure of the microscopic objects can be seen by secondary electron measurement. By reflected electron measurement, it is seen that electrons reflected by the produced microscopic objects have almost a uniform luminance.

According to a method for producing a hollow microscopic object in another embodiment according to the present invention, on a surface of a specific layer among at least two thin film layers included in a hollow microscopic object, nanometer-sized microparticles of a substance different from the substance used for the thin film layers are located in a dispersed manner. The microscopic object produced in this manner has a structure in which the microparticles are embedded two-dimensionally in a dispersed manner at an interface sandwiched between two specific layers among the at least two layers (namely, the microparticles are expanded in a direction parallel to the inter-layer interface).

This will be described more specifically. For example, after the step of depositing at least one layer of a transition metal, a metal or a semiconductor described above, the step of dispersing the microparticles on the surface of the deposited layer to bind the microparticles to the surface and the step of, subsequently, depositing again at least one layer a transition metal, a metal or a semiconductor again are performed. As a result, the microparticles are embedded two-dimensionally in a dispersed manner at an interface sandwiched between the substance deposited to form the first layer and the substance deposited to form the second layer.

As an example, a method of holding quantum dots in a microscopic object to control the optical characteristics will be described. Quantum dots are microscopic particles formed of an element such as cadmium-selenium or the like and having a diameter of several nanometers. Quantum dots have a feature of emitting fluorescence depending on the size thereof. In the field of biology, quantum dots are widely used as a label of an intended biological molecule. It is known that the fluorescence blinks or is extinguished due to an ambient environmental factor such as the distance between adjacent quantum dots, the solvent condition or the like. It is also known that when two types of quantum dots emitting light of different wavelengths are located within a certain distance, a resonance phenomenon occurs because of energy transfer and light of a discriminative wavelength is emitted.

In another embodiment, the microparticles may be formed of any of ferromagnetic single-substance elements exhibiting a ferromagnetic property at room temperature such as iron, cobalt, nickel, gadolinium and the like; magnetic oxides referred to as “ferrites” such as FeO.Fe₂O₃, MnO.Fe₂O₃, NiO.Fe₂O₃, CoO.Fe₂O₃ and the like; and ferrimagnetic materials including insulating ferrimagnetic materials such as iron garnet (garnet) M₃.Fe₅O₁₂ (M represents an element of Fe, Y, Mn or the like), yttrium iron garnet (YIG) Y₃.Fe₅O₁₂ and the like. When being used for the microparticles, such a substance may be put into a microscopic size too small to maintain a magnetic domain structure and located between two layers in a dispersed manner so as not to contact each other. In this manner, a hollow microscopic object including such microparticles can have a superparamagnetic property. The microscopic size may be preferably set to 5 nm or less so that a superparamagnetic property which is guaranteed to fail in maintaining a magnetic domain structure is realized.

Using the microscopic object provided by the present invention, the optical characteristics can be controlled by controlling the distance between the quantum dots. FIG. 10 shows a method for the control. As the element 1 used to form the microscopic object, a material which is easily chemically modified such as gold or the like is used. The quantum dots 7 are immobilized to the element 1. The immobilization may be performed by, for example, a method of using construction of a two-dimensional spatial structure of DNA, a method of introducing an amino group, a thiol group or biotin to both of two termini of DNA to perform crosslinking, a method using protein such as BSA, antibody or the like, or a method of using a double crosslinker. After the quantum dots 7 are immobilized to a surface of the element 1, a layer of the same type of element, namely, element 1′, is stacked to confine the quantum dots 7 in the element 1. This allows the distance between adjacent quantum dots to be controlled. In addition, the confinement allows a certain ambient environment of the quantum dots 7 to be maintained and thus to allow a stable fluorescence to be emitted.

Next, a layer of a different type of element, namely, element 2, is stacked, and quantum dots 8 exhibiting optical characteristics different from those of the quantum dots 7 are immobilized to a surface of the element 2. Then, a layer of the same type of element, namely, element 2′, is stacked to confine the quantum dots 8. A total thickness of the element 1′ and the element 2 is set to an appropriate value between 2 nm to 1 μm to cause a fluorescence resonance phenomenon between the quantum dots 7 and 8 and thus to stably emit fluorescence having a discriminative wavelength. As the element 2, a substance which can be eluted in the case where the solvent is, for example, aluminum or copper is used, so that the elements 2 and 2′ can be removed, and also the quantum dots 8 can be removed along with the elements 2 and 2′, from the microscopic object. As a result, the fluorescence emitted from the microscopic object has a wavelength of the fluorescence which is emitted by the quantum dots 7. Utilizing this, the microscopic object is usable as a sensor which emits light of different wavelengths in accordance with the solvent conditions.

The microscopic object provided by the present invention is also usable as a microscopic trap for capturing target biological molecules. FIG. 11 shows a method for capturing the target biological molecules. As shown in FIG. 11(a), hollow microscopic objects 9 each including one or a plurality of layers are each located such that an opening thereof is directed opposite to a substrate 6. The microscopic objects 9 are located in this manner as follows. An adhesive tape or the like is used as a material of the substrate 6. The adhesive tape is gently bonded to top surfaces of the hollow microscopic objects produced as described above with reference to FIG. 8, and the hollow microscopic objects with the adhesive tape are gently peeled off from the substrate on which the hollow microscopic objects are arranged. Next, biological molecules 10 to be reacted with target biological molecules 11 are immobilized to an inner surface of each microscopic object 9. The inner surfaces of the hollow microscopic objects 9 are formed of gold, silver or the like, so that the biological molecules 10 are immobilized thereto via an amino group or a thiol group contained in the protein. An electric field is applied between substrate 6 and another substrate 6′ located in a solution containing the target biological molecules 11 dispersed therein. As a result, target biological molecules can be captured inside the hollow microscopic objects. With this method, in the case where, for example, the biological molecules 10 for reaction are of antibody and the target biological molecules 11 are of antigen, only the antigen can be selectively recovered from the solvent. In the case where the biological molecules 10 for reaction are of chaperone protein and the target biological molecules 11 are of protein having a structure thereof destroyed, the intended protein can be confined in the inner spaces of the hollow microscopic objects and thus repaired efficiently.

According to another method, as shown in FIG. 11(b), chain-like biological molecules 14 are immobilized inside the hollow microscopic objects 9 and the chains are extended or folded to recover target biological molecules 12. The chain-like biological molecules 14 may be of DNA, macromolecular polymer, or those formed of these molecules that are coupled to each other by a molecule having a structure changeable by light irradiation and/or electric field application. The molecule having a structure changeable by light irradiation, electric field application, magnetic field application or the like is, for example, diazobenzene, the structure of which is changed by being irradiated with light having a wavelength of 365 nm. To the chain-like biological molecules 14, capturing biological molecules 13 for capturing the target biological molecules 12 are immobilized in advance. The immobilization may be performed by chemically crosslinking side chains of the chain-like biological molecules 14 and an amino group, a thiol group or a carboxyl group of the capturing biological molecules 13. The chain-like biological molecules 14 are extended to be straight by electric and/or magnetic field application, light irradiation or change in the salt concentration of the solvent to bind the target biological molecules 12 in the solvent to the capturing biological molecules 13. Next, the chain-like biological molecules 14 are folded inside the hollow microscopic objects by changing the application of the electric and/or magnetic field or the salt concentration of the solvent in the opposite direction, or stopping the light irradiation to recover the target biological molecules 12 inside the hollow microscopic objects 9. The biological molecules 10 for reaction are immobilized inside the hollow microscopic objects 9 in advance, so that the target biological molecules 12 and the biological molecules 10 for reaction are efficiently reacted with each other inside the microscopic objects. After the reaction, the chain-like biological molecules 14 are extended by re-applying the electric and/or magnetic field, resuming the light irradiation, or changing the salt concentration of the solvent again to put the target biological molecules 12 back into the solvent. In the case where, for example, the biological molecules 10 for reaction are of chaperone protein, the target biological molecules 12 are of protein having a structure thereof destroyed, and the capturing biological molecules 13 are of antibody to the protein, the protein can be repaired efficiently.

According to still another method, the hollow microscopic objects 9 are not immobilized to the substrate 6. Instead, as shown in FIG. 11(c), two hollow microscopic objects are coupled to each other by a chain-like biological molecule 14 to capture the target biological molecules 12 contained in the solvent. The two hollow microscopic objects may be coupled to each other as follows. The inner surface of each of the microscopic objects is formed of gold or silver, and an amino group, a thiol group or a carboxyl group is introduced into both of two termini of the chain-like biological molecule 14 and the biological molecules are mixed with the microscopic objects. The chain-like biological molecule 14 is extended to be straight by electric or magnetic field application, light irradiation, or change in the salt concentration of the solvent so as to separate the two hollow microscopic objects 9 from each other and thus to expose capturing biological molecules 13. As a result, the target biological molecules 12 in the solvent are bound to the capturing biological molecules 13. Next, the chain-like biological molecule 14 is folded inside the hollow microscopic objects 9 by changing the application of the electric or magnetic field or the salt concentration of the solvent in the opposite direction, or stopping the light irradiation. As a result, the two hollow microscopic objects 9 are assembled together into a closed state to capture the target biological molecules 12 in the space formed by the hollow microscopic objects 9. The chain-like biological molecule 14 may be extended by re-applying the electric and/or magnetic field, resuming the light irradiation, or changing the salt concentration of the solvent again to put the target biological molecules 12 back into the solvent.

The hollow microscopic objects can be provided with a superparamagnetic property by being supplied with a layer that is formed of an element or a compound having a ferromagnetic property at room temperature and has a thickness too small to maintain a magnetic domain structure, or by being supplied with microparticles, held between the layers, having a particle size too small to maintain a magnetic domain structure. Usable for such microparticles are, specifically, ferromagnetic single-substance elements exhibiting a ferromagnetic property at room temperature such as iron, cobalt, nickel, gadolinium and the like; magnetic oxides referred to as “ferrites” such as FeO.Fe₂O₃, MnO.Fe₂O₃, NiO.Fe₂O₃, CoO.Fe₂O₃ and the like; and ferrimagnetic materials including insulating ferrimagnetic materials such as iron garnet (garnet) M₃.Fe₅O₁₂ (M represents an element of Fe, Y, Mn or the like), yttrium iron garnet (YIG) Y₃.Fe₅O₁₂ and the like.

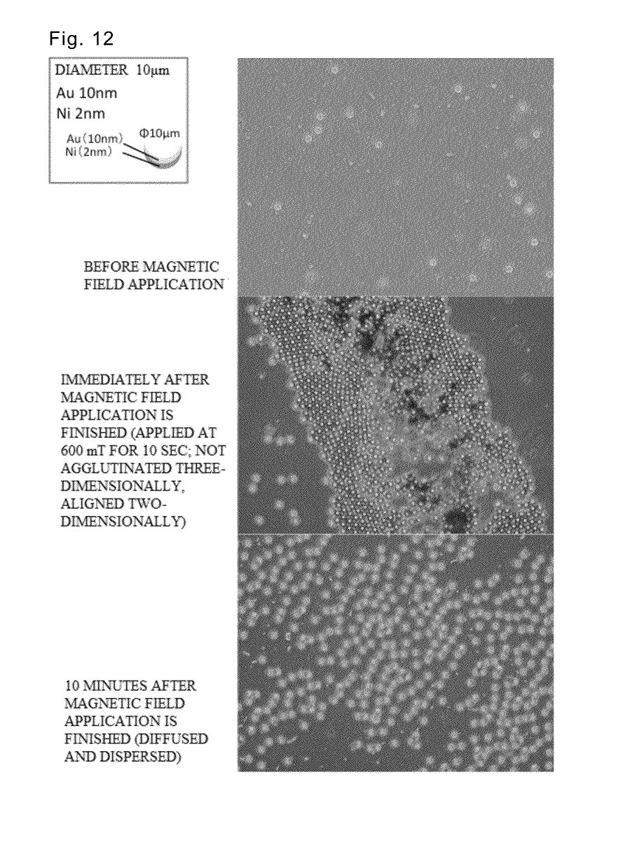

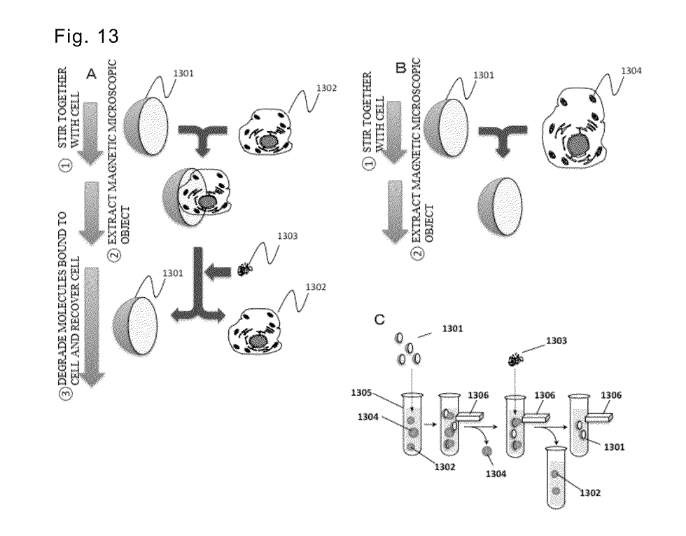

FIG. 12 shows the states of hollow microscopic objects including a ferromagnetic layer having a thickness too small to maintain a magnetic domain structure, before, during and after the application of an external magnetic field. As shown in the figure, the hollow microscopic objects each include two layers. The outer layer is formed of nickel, which is a ferromagnetic metal, and the inner layer is formed of gold. The hollow microscopic objects each have a diameter of 10 μm, the gold inner layer has a thickness of 10 nm, and the nickel outer layer has a thickness of 2 nm. An external magnetic field was applied to the hollow microscopic objects, and the response thereof was observed. It was confirmed that when a magnet (neodymium, 0.6 T) was put close to the microscopic objects, the microscopic objects were attracted to each other and gathered; whereas when the magnet was put far therefrom to remove the external magnetic field, the microscopic objects were dispersed. Based on this, it can be confirmed that in the case where the thickness of the nickel layer is 2 nm, the magnetic domain structure cannot be maintained, namely, the microscopic objects have a superparamagnetic property. By contrast, in the case where the diameter of each microscopic object was 10 μm and the thickness of the gold inner layer was 10 nm as in FIG. 12 but the thickness of the nickel outer layer was 5 nm, the results were as follows. When an external magnetic field was applied by use of a magnet and then was removed, some of the microscopic objects were kept agglutinated. Based on this, the nickel layer thickness of 5 nm is the border between a state where the microscopic objects are superparamagnetic and cannot maintain the magnetic domain structure and a state where the microscopic objects are ferroparamagnetic and can construct the magnetic domain structure.

FIG. 13 schematically shows exemplary methods for actually recovering a cell using a magnetic hollow microscopic object having a superparamagnetic property at room temperature. FIG. A schematically shows how a cell 1302 having a size smaller than that of a hemispherical magnetic hollow microscopic object 1301 interacts with the microscopic object 1301 and are recovered and purified. First, in stirring step (i), in the case where a cell interactive with a target cell such as DNA aptamer, antibody or the like that modifies an inner surface of the microscopic object 1301 is sufficiently small to be physically contactable with the inner surface of the microscopic object 1301, such a cell can interact with a surface of the target cell to bind the cell 1302 and the microscopic object 1301 to each other. Next, in step (ii), such microscopic objects are agglutinated by an external magnetic field by use of a permanent magnet 1306 or the like, and the cells which are not bound to the microscopic objects 1301 are removed to leave only the microscopic objects 1301 and the target cells 1302. Then, a factor that immobilizes the cell to the inner surface of each microscopic object 1301, such as a DNA degrading enzyme 1303 or the like, is degraded. Alternatively, as described regarding the above-described technique, the microscopic object may be degraded. Next, only the microscopic object 1301 is recovered again by use of the permanent magnet 1306, and thus only the target cell 1302 can be recovered. FIG. B shows an example in which a cell 1304 has a size larger than that of the inner space of the hollow microscopic object 1301. As can be seen from the figure, the cell 1304 cannot contact a modification macromolecule or the like that is interactive with the cell on the inner surface of the microscopic object 1301. Therefore, even if the microscopic object 1301 is recovered by use of the permanent magnet 1306, the cell 1304 is not recovered. FIG. C schematically shows a procedure in which a test tube 1305 is actually used. The microscopic objects 1301 are put into the test tube 1305 containing various cells 1302 and 1304, and the solution is replaced while the microscopic objects 1301 are attracted to the permanent magnet 1306. As a result, the cells 1304 which are not bound to the microscopic objects 1301 are removed. Next, in the case where DNA aptamer is used as a target binding substance, a substance that degrades a cell binding substance, such as a DNA degrading enzyme 1303 or the like, is added, and the microscopic objects 1301 are removed by use of, again, the permanent magnet 1306. In this manner, the cells 1302, which are the targets of recovery, can be recovered.

In this example, the microscopic objects 1301 of a uniform size are used. The steps shown in FIG. 13 may be performed in repetition for a plurality of sizes of microscopic objects, sequentially from larger-sized microscopic objects to smaller-sized microscopic objects, so that size fractionation can be performed.

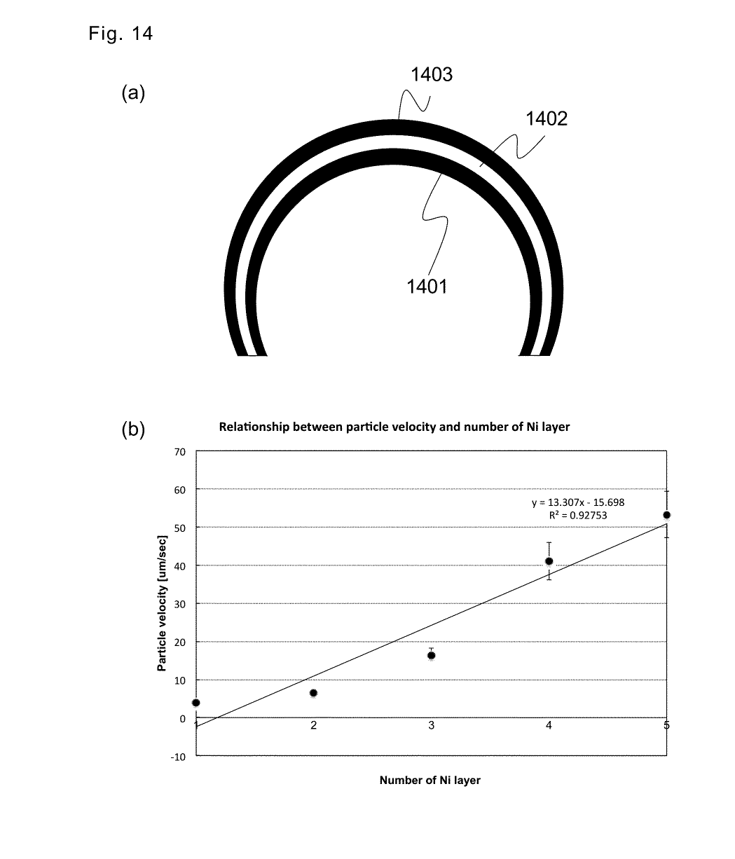

In order to improve the responsiveness of a microscopic object having a superparamagnetic property to an applied magnetic field, the microscopic object may be produced with a plurality of (at least two) magnetic layers. FIG. 14(a) schematically shows a simplest example of such a microscopic object. The microscopic object includes two ferromagnetic layers (1401, 1403) each having a thickness too small to maintain a magnetic domain structure, and also includes an insulating layer (1402) that separates the ferromagnetic layers (1401, 1403). Elements and compounds usable to form the insulating layer include oxides such as SiO, SiO₂, manganese oxide, nickel oxide, alumina and the like; polymers such as Teflon (registered trademark) and the like; organic polymers such as plastics and the like; and insulators. The thickness of the insulating layer that separates the magnetic layers from each other, which depends on the type of the substance, is generally about 5 nm or more, and preferably about 10 nm or more. With such a thickness of the insulating layer, the multiple layer structure in which the magnetic layers are separated from each other by the insulating layer can have a superparamagnetic property. A preferable non-limiting example thereof is as follows. On a polystyrene spherical mold having a diameter of 10 μm, an Ni layer was formed to a thickness of 2 nm, then an SiO₂ layer was formed thereon to a thickness of 20 nm, and another Ni layer was formed thereon to a thickness of 2 nm. These steps were repeated to produce a microscopic object including one, two, three, four or five Ni layers. Then, a magnet (neodymium, 0.6 T) was put close to each of the microscopic objects dispersed in water containing 0.1% Tween20. On the stage where a force by which each microscopic object was attracted to the magnetic field was equalized to the viscous resistance of the solvent and thus the velocity was made constant, the moving rate of each microscopic object was measured by microscopic observation. FIG. 14(b) is a graph showing the relationship between the number of the magnetic layers included in the microscopic object and the response (moving) rate of the microscopic object to the application of a magnetic field. As shown in FIG. 14(b), as the number of the magnetic layers included in the microscopic object increases, the response rate to the application of a magnetic field increases generally linearly. Based on this, it is seen that in the case where rapid recovery by application of a magnetic field is wished, it is desirable to produce a microscopic object including a large number of magnetic layers; whereas in the case where gentle recovery is wished, it is desirable to produce a microscopic object including a small number of magnetic layers.

### INDUSTRIAL APPLICABILITY

As described above, according to the present invention, a hollow microscopic object formed of only a desired element can be produced by depositing an intended element on an organic mold arranged on a substrate and then decomposing and thus removing the organic mold. Utilizing this, various types of microscopic objects can be produced in a large number. Therefore, various types of biological molecules can be labeled at the same time, and also novel materials can be developed by use of microscopic objects formed of various elements of a nanometer size.

A hollow microscopic object and a method for recovering a biological substance or the like using the same according to the present invention are useful to recover, selectively and/or effectively, a target microparticle such as a cell or the like of a specific size. This replaces filter fractionation. In the case where at the time of production of the microscopic object, the material, thickness and number of the element layers is optimally selected, the recovery is allowed to be performed rapidly or gently. A microscopic object having a size of 1 μm or less is useful to recover, selectively and/or effectively and in a size-dependent manner, a small target microparticle such as an intra-cell structure or the like, which cannot be easily recovered conventionally.

### REFERENCE SIGNS LIST

1 . . . element 1; 1′ . . . deposited layer of the same material as element 1; 2 . . . element 2; 2′ . . . deposited layer of the same material as element 2; 3 . . . element 3; 4 . . . organic mold; 4′ . . . organic mold decomposed and thus removed by an ultraviolet-ozone process; 5-1 . . . vapor deposition source for depositing the element 1; 5-2 . . . vapor deposition source for depositing the element 2; 6 . . . substrate 1; 6′ . . . substrate 2; 7 . . . quantum dot 1; 8 . . . quantum dot 2; 9 . . . hollow microscopic object including one or a plurality of layers; 10 . . . biological molecule 1; 11 . . . biological molecule 2; 12 . . . biological molecule 3; 13 . . . biological molecule for capturing 12; 14 . . . chain-like biological molecule; 1301 . . . magnetic hollow microscopic object; 1302 . . . cell having a size sufficiently small to be put on an inner surface of the microscopic object; 1303 . . . DNA aptamer degrading enzyme; 1304 . . . cell having a size too large to be put on an inner surface of the microscopic object; 1305 . . . test tube; 1306 . . . permanent magnet; 1401 . . . magnetic layer; 1402 . . . insulating layer; 1403 . . . magnetic layer.

1. A hollow microscopic object comprising: a layer structure which forms an outer shell and comprises at least one thin film layer of a transition metal, a metal or a semiconductor; and an inner space and an opening defined by the layer structure, wherein:
the layer structure includes:
at least two thin film layers; and
microparticles embedded two-dimensionally in a dispersed manner at an interface between the at least two thin film layers; and
the microparticles are formed of a substance which exhibit a ferromagnetic property or a ferrimagnetic property at room temperature or a quantum dot, have a size too small to maintain a magnetic domain structure and are formed of a substance different from that of the thin film layers.

2. A hollow microscopic object comprising: a layer structure which forms an outer shell and comprises at least one thin film layer of a transition metal, a metal or a semiconductor; and an inner space and an opening defined by the layer structure, wherein:
the layer structure includes at least two thin film layers; and
at least one of the layers is formed of a substance exhibiting a ferromagnetic property or a ferrimagnetic property at room temperature and has a thickness too small to maintain a magnetic domain structure.

3. A hollow microscopic object comprising: a layer structure which forms an outer shell and comprises at least one thin film layer of a transition metal, a metal or a semiconductor; and an inner space and an opening defined by the layer structure, wherein:
the layer structure includes at least three thin film layers; and
at least two of the layers are formed of a substance exhibiting a ferromagnetic property or a ferrimagnetic property at room temperature and have a thickness too small to maintain a magnetic domain structure, and a layer that separates the at least two layers from each other is formed of an insulator and has a thickness that provides such a distance between the magnetic layers as not to generate a ferromagnetic property.

4. The microscopic object according to claim 1, wherein the transition metal has any of atomic numbers up to 79 except for 43, the metal has any of atomic numbers 13, 31, 32, 33, 49, 50, 51, 81, 82 and 83, and semiconductor has any of atomic numbers 14, 34 and 52, of the periodic table.

5. The microscopic object according to claim 1, wherein:
the substance which exhibits a ferromagnetic property at room temperature is selected from the group consisting of iron, cobalt, nickel and gadolinium; and
the substance which exhibits a ferrimagnetic property at room temperature is selected from the group consisting of FeO.Fe₂O₃, MnO.Fe₂O₃, NiO.Fe₂O₃, CoO.Fe₂O₃, iron garnet (garnet) M₃.Fe₅O₁₂ (M represents an element of Fe, Y, Mn or the like), and yttrium iron garnet (YIG) Y₃.Fe₅O₁₂.

6. The microscopic object according to claim 1, wherein:
(i) an outermost layer of the layer structure is formed of gold and has a thickness of 2 nm or more; or
(ii) an innermost layer of the layer structure is formed of gold and has a thickness of 2 nm or more.

7. The microscopic object according to claim 3, wherein:
(i) the layers separated from each other by the insulator which does not have a ferromagnetic property or a ferrimagnetic property at room temperature each have a thickness of 10 nm or more; or
(ii) the layers separated from each other by the insulator which does not have a ferromagnetic property or a ferrimagnetic property at room temperature are each formed of an insulating substance or a metal oxide.

8. The microscopic object according to claim 2, wherein a layer of the microparticles has a thickness of 5 nm or less.

9. The microscopic object according to claim 1, wherein the quantum dot is selected from the group consisting of Cds, CdSe, CdTe, CdHgTe and HgTe.

10. The microscopic object according to claim 1, wherein:
the layer structure includes at least two thin film layers;
an outermost thin film layer of the layer structure is formed of a substance soluble by a predetermined liquid; and
an inner thin film layer of the layer structure is formed of a substance insoluble by the liquid.

11. The microscopic object according to claim 10, wherein the outermost thin film layer is formed of a metal, and the inner thin film layer is formed of a dielectric substance or a semiconductor.

12. The microscopic object according to claim 1, further comprising a macromolecule having one end thereof immobilized to a surface of the layer structure that is exposed to the inner space.

13. The microscopic object according to claim 12, wherein an innermost layer and an outermost layer of the layer structure are formed of different substances from each other, the substance of the innermost layer is suitable to allow for the one end of the macromolecule to be attached thereto, and the substance of the outermost does not easily allow the one end of the macromolecule to be attached thereto.

14. The microscopic object according to claim 12, wherein the innermost layer is formed of any one of gold, silver, silicon and silicon oxide, and the outermost layer is formed of any one of iron, copper, germanium, aluminum, chromium, tin, titanium, manganese, nickel, cobalt and gadolinium.

15. The microscopic object according to claim 12, wherein the macromolecule has a structure thereof changeable by change in ionic strength and/or pH of a solution, electric field application, magnetic field application, or light irradiation.

16. The microscopic object according to claim 12, wherein the macromolecule is a DNA chain or cellulose polymer, or those formed of a DNA chain and a cellulose polymer that are coupled to each other by a molecule having a structure thereof changeable by electric field application, magnetic field application or light irradiation.

17. The microscopic object according to claim 12, wherein the macromolecule is, for example, a nucleic acid molecule or molecular chain, a nucleic acid derivative molecule or molecular chain, a molecular chain of protein such as antibody or the like, a macromolecular chain bindable to a cell surface or the like.

18. The microscopic object according to claim 12, wherein the other end of the macromolecule is immobilized to another microscopic object or a surface of another substrate.

19. A method for producing a hollow microscopic object, comprising the step of depositing at least one thin film layer of a transition metal, a metal or a semiconductor on a mold, which is a microstructure formed of an organic substance, to form a layer structure, wherein:
(i) the method further comprises, after the step of depositing at least one thin film layer of a transition metal, a metal or a semiconductor, the step of dispersing microparticles on a surface of the deposited layer to bind the microparticles thereto, and the step of, subsequently, depositing again at least one layer of a transition metal, a metal or a semiconductor, thereby embedding the microparticles two-dimensionally in a dispersed manner at an interface sandwiched between the substance deposited to form the first layer and the substance deposited to form the second layer; and
the microparticles are formed of a substance which exhibits a ferromagnetic property or a ferrimagnetic property at room temperature or a quantum dot, have a size too small to maintain a magnetic domain structure and are formed of a substance different from that of the thin film layers; or
(ii) the layer structure includes at least two thin film layers; and at least one of the layers is formed of a substance exhibiting a ferromagnetic property or a ferrimagnetic property at room temperature and has a thickness too small to maintain a magnetic domain structure; or
(iii) the layer structure includes at least three thin film layers; and at least two of the layers are formed of a substance exhibiting a ferromagnetic property or a ferrimagnetic property at room temperature and have a thickness too small to maintain a magnetic domain structure, and a layer that separates the magnetic layers from each other is formed of an insulator.

20. A method for producing a hollow microscopic object containing a transition metal, a metal or a semiconductor, the method comprising the steps of:
dripping an organic mold suspension, containing organic molds having a predetermined diameter, an appropriate amount of pure water, and a material for suppressing a static repulsive force between the organic molds, onto one surface of a substrate to distribute the organic molds on the substrate at a predetermined density;
washing and thus removing an excessive amount of the organic molds that is not adsorbed to the substrate;
drying the organic molds distributed on the substrate;
cutting the organic molds to adjust a gap between the organic molds arranged on the substrate to a predetermined distance;
depositing at least one thin film layer of a transition metal, a metal or a semiconductor on the organic molds distributed on the substrate; and
decomposing and thus removing the organic molds having the at least one thin film layer of a transition metal, a metal or a semiconductor deposited thereon to obtain each of the hollow microscopic objects which are left above the substrate;
wherein:
(i) the method further comprises, after the step of depositing at least one thin film layer of a transition metal, a metal or a semiconductor, the step of dispersing microparticles on a surface of the deposited layer to bind the microparticles thereto, and the step of, subsequently, depositing again at least one layer of a transition metal, a metal or a semiconductor, thereby embedding the microparticles two-dimensionally in a dispersed manner at an interface sandwiched between the substance deposited to form the first layer and the substance deposited to form the second layer;
the microparticles are formed of a substance which exhibits a ferromagnetic property or a ferrimagnetic property at room temperature, has a size too small to maintain a magnetic domain structure and is different from that of the thin film layers; or
(ii) the thin film layer includes at least two thin film layers; and at least one of the layers is formed of a substance exhibiting a ferromagnetic property or a ferrimagnetic property at room temperature and has a thickness too small to maintain a magnetic domain structure; or
(iii) the layer structure includes at least three thin film layers; and at least two of the layers are formed of a substance exhibiting a ferromagnetic property or a ferrimagnetic property at room temperature and have a thickness too small to maintain a magnetic domain structure, and a layer that separates the magnetic layers from each other is formed of an insulator.

21. The method according to claim 20, wherein the organic molds are cut by any one of a plasma etching process, an ion milling process, a converged ion beam process, and a resist process.

22. The method according to claim 20, wherein the step of depositing at least one thin film layer of a transition metal, a metal or a semiconductor on the organic molds distributed on the substrate is performed by any one of a resistive heating vacuum vapor deposition method, a sputtering method, and a chemical vapor deposition method.

23. The method according to claim 20, wherein the step of decomposing and thus removing the organic molds having the at least one thin film layer of a transition metal, a metal or a semiconductor deposited thereon is performed by any one of an ultraviolet-ozone process, a plasma decomposition process, a photocatalyst decomposition process, and a heating and incineration process.

24. The method according to claim 20, wherein the step of obtaining each of the hollow microscopic objects further includes:
dripping a microscopic amount of liquid onto each of the hollow microscopic objects; and
while allowing ultrasonic waves to act on the other surface of the substrate, placing a member having a flat bottom surface on the one surface to which the hollow microscopic objects are immobilized such that a slight load is applied to the bottom surface, and moving the member in an optional direction to delaminate the microscopic objects from the substrate.

25. The method according to claim 24, wherein the liquid dripped onto the hollow microscopic objects is pure water, or a combination of pure water and protein such as bovine serum, antibody or bovine serum albumin (BSA); synthetic DNA; or a surfactant such as citrate, phosphate, sodium dodecyl sulfate (SDS) or tannic acid.

26. The method according to claim 19, wherein the substrate is a silicon substrate, a glass substrate, an aluminum substrate, or a plastic substrate.

27. A method for recovering a biological substance by use of a hollow microscopic object having an inner surface modified by a substance specifically bindable to a specific target biological substance and having a superparamagnetic property, the method comprising the steps of:
mixing the hollow microscopic object and a solution containing the target biological substance; and
attracting the substance that modifies the inner surface of the hollow microscopic object and is bindable to the target biological substance and also attracting the target biological substance bound thereto, by use of an external magnetic field, and recovering the substance and the target biological substance by a magnetic power.

28. The method according to claim 27, wherein the biological substance is cell.

29. The method according to claim 27, further comprising the step of, after the step of recovering, degrading the substance bound to the target biological substance and recovering the target biological substance.

30. The method according to claim 29, wherein DNA aptamer is used as the substance bindable to the target biological substance, and a DNA degrading enzyme is used in the step of degrading.

31. The method according to claim 27, further comprising the step of, after the step of recovering, degrading the hollow microscopic object.

32. The method according to claim 27, wherein the hollow microscopic object is a hollow microscopic object according to any one of claims 12 through 18.

33. The method according to claim 27, wherein the size of the hollow microscopic object is adjusted to allow size fractionation to be performed on the target biological substance.
